(19) United States
(12) Patent Application Publication
(10) Pub. No.: US 2004/0133951 A1
(43) Pub. Date: Jul. 8, 2004

(54) Method and materials for introgression of novel genetic variation in maize

(76) Inventor: Mary Wilkes Eubanks, Durham, NC (US)

Correspondence Address: Mary Wilkes Eubanks, 8 Pilton Place, Durham, NC, 27705, US

(21) Appl. No.: 10/614,255

(22) Filed: Jul. 3, 2003

Related U.S. Application Data
(63) Continuation-in-part of application No. 09/368,869, filed on Aug. 5, 1999, now Pat. No. 6,617,492.

(51) Int. Cl. C12Q 1/68; A01H 5/00

(52) U.S. Cl. 800/320.1; 435/6

(57) ABSTRACT

There is provided a method for transferring novel genetic materials into maize by crossing (Tripsacum X perennial Zea diploperennis) with maize. This invention thus relates to the novel genetic materials in the seed, plants produced by the seed and/or tissue culture, variants, mutants, modifications, and cellular and molecular components of Tripsacum-Z. diploperennis hybrids and of hybrids between Tripsacum-Z. diploperennis and maize. In particular this invention is directed to the ability to transfer nucleotide sequences and novel alleles into maize for genetic analyses and selection of valuable agronomic traits including: resistance to insects and diseases including European corn borer and aflatoxin; tolerance to drought, cold, flooding, corn rootworm, acid soils, low nitrogen; apomixis, totipotency, perennialism and ability to produce double haploids; adaptation to adverse soil conditions; more extensive root systems with aerenchyma and strong capacity for regrowth; enhanced grain quality and nutrition.

### RELATED U.S. PATENT APPLICATION DATA

This is a Continuation-in-part of U.S. Ser. No. 09/368,869 filed Aug. 5, 1999, Notice of Allowance 14 Apr. 2003.

### FEDERALLY SPONSORED RESEARCH

Part of the research on which this patent application is based was funded by National Science Foundation Grants No. 9660146 and 9801386.

### FIELD OF THE INVENTION

This invention relates generally to the fields of molecular genetics, cytogenetics, and plant breeding. More particularly, it relates to a method for identifying allelic fragments of DNA that correspond to genomic introgression segments of Tripsacum origin, and/or recombinant segments of Tripsacum-teosinte chimeric genetic origin, which are stably inherited in wide cross hybrids between Tripsacum and teosinte (i.e. wild Zea sp.) and the progeny from crosses between maize and Tripsacum-teosinte recombinants. These unique DNA fragments are distinct because they are not found in the Zea parent, maize (Zea mays L.), or teosinte species. Maize is the common name used around the world for the staple cereal grain generally referred as corn in the United States. The two terms are used interchangeably throughout this document. These variant DNA fragments formed as a result of intergeneric hybridization between gamagrass (Tripsacum sp.) and teosinte (Zea sp.) provide unique markers for assisting selection of desirable traits in maize breeding programs, for detection of target DNA sequences in genetic analyses, and for the identification and transfer of new genes in corn improvement that confer resistance to insect pests and diseases, drought stress tolerance, cold tolerance, perennialism, increased grain yield, totipotency, apomixis, improved root systems, root aerenchyma, ability to grow in anaerobic conditions, tolerance of water-logged soils, tolerance of high-aluminum and acidic soils, improved grain quality, enhanced forage quality, ability to attract nitrogen-fixing bacteria to the rhizoshpere, herbicide tolerance, toxic metal tolerance, adaptability to a CO2 enriched atmosphere and other environmental stresses.

### BACKGROUND OF THE INVENTION

Molecular Genetics.

Genetics is the study of genes and heritable traits in biological organisms. In plant breeding, the goal of molecular genetics is to identify genes that confer desired traits to crop plants, and to use molecular markers (DNA signposts that are closely associated with specific genes) to identify individuals that carry the gene or genes of interest in plants (Morris 1998), to determine the DNA sequences and characterize gene expression and function. A genetic marker is a polymorphism or variant allele that reveals the genetic locus or loci in an individual genotype that is associated with the phenotypic expression of a morphological or anatomical characteristic or a biochemical or physiological process. Variants in DNA and proteins are used as markers in molecular genetics. Molecular variants, or mutant genes, are used in genetic analysis to identify a particular gene and its functional role in precise biological activities and processes. Mutation is the process whereby nucleotide sequences and genes change from the reference form, generally designated wild type, to a different form. In genetic analysis mutants are a valuable source of genotypic variation that allows selection of new phenotypes (Griffiths et al. 1993). Mutations occur either in a sequence of nucleotides, a gene (i.e. DNA sequence that codes for a phenotypic trait), or in the chromosomes (i.e. the hereditary packages that contain specific DNA nucleotide sequences supercoiled with proteins into individual units that can be observed cytologically in the nucleus of a cell, also referred to as linkage groups). In one type of mutation, the nucleotides that comprise the wild type allele of a gene (i.e. the established reference form that marks a particular locus on a chromosome) are altered resulting in a point mutation. In chromosome mutations, segments of chromosomes, whole chromosomes, or entire sets of chromosomes are rearranged via inversions, translocations, fusions or deletions.

The dogma is that mutations are rare, and most newly formed mutations are deleterious. Data on mutation frequencies for seven genes in maize provide a baseline indicating the rarity of mutations in maize (Stadler 1951). Mutation frequency ranged from 0.000492% (i.e. 492 mutants out of a million gametes) in the red color(R) gene; 0.000106% (i.e. 106 out of a million) for the inhibitor of R (I) gene; 0.000011% (i.e. 11 out of a million) for the purple aleurone (Pr) gene; 0.0000024% (i.e. 2.4 out of a million) for the starchy (Su) gene; 0.0000022% (i.e. 2.2 out of a million) for the yellow color (Y) gene; 0.0000012% (i.e. 1.2 out of a million) for the normal kernel (Sh) gene, and 0% (i.e. 0 out of a million for the waxy gene (Wx) .

Because of the rarity of spontaneous mutations, geneticists and plant breeders typically use mutagens such as chemicals and radiation to increase the frequency of mutation rates in order to increase the number of variant forms that might be useful for genetic analysis and selection of new traits. Another method of inducing mutagenesis in maize is transposon tagging whereby a maize line is crossed with a line containing one of the three systems of transposable elements found in maize. When a transposable element inserts into a gene, it causes a mutation. The reported mutation frequencies for transposable element mutator lines varies from 1 in a thousand to 1 in a million (Chomet 1994). To find a mutation in maize using one of these mutagenic lines, a breeder must screen a minimum of 100,000 plants.

Plant Breeding.

Conventional plant breeding is the science that utilizes crosses between individuals with different genetic constitutions. The resulting recombination of genes between different lines, families, species, or genera produces new hybrids from which desirable traits are selected. Plant breeding is achieved by controlling reproduction. Since maize is a sexually reproducing plant, techniques for controlled pollination are frequently employed to obtain new hybrids. Controlling reproduction in maize involves continually repeating two basic procedures: (1) evaluating a series of genotypes, and (2) self-pollinating or crossing among the most superior plants to obtain the next generation of genotypes or progeny. Controlled pollinations in maize utilize two procedures: (1) detasseling, and (2) hand pollination.

Hybrid corn results from crossing specifically selected parental strains called “inbred lines” (Griliches 1957). The inbreds are produced by self-pollinating for a few years to obtain plants that uniformly express desired traits being selected by the breeder. Inbreds themselves, which are less vigorous due to inbreeding depression, are not suitable for the commercial market. However, when two inbred lines from different heterotic groups are crossed, hybrid vigor develops and the resulting hybrids are far superior to the original varieties. This is the process for development and production of commercial hybrid corn seed.

Maize is a monoecious grass that has separate male and female flowers on the same plant. The male or staminate flowers produce pollen in the tassel at the apex of the maize stalk, and the female or pistillate flowers that produce the grain when pollinated are borne laterally in leaf axils tangential to the stalk. Pollination is accomplished by transfer of pollen from the tassel to silks which emerge from the axillary pistillate ears. Since maize is wind-pollinated, controlled pollination in which pollen is collected from the tassel of one plant and transferred by hand to the silks of the same or another plant, is a technique used in maize breeding. The steps involved in making controlled crosses and self-pollinations in maize are standard practice (Neuffer 1982) and are as follows: (1) the ear emerging from the leaf shoot is covered with an ear shoot bag one or two days before the silks emerge to prevent contamination by stray pollen; (2) prior to making a pollination, the ear shoot bag is quickly removed and the silks cut with a knife to form a short brush, then the bag is immediately placed back over the ear; (3) also prior to making a pollination, the tassel is covered with a tassel bag to collect pollen; (3) on the day crosses are made, the tassel bag with the desired pollen is carried to the plant for crossing, the ear shoot bag is removed and the pollen dusted on the silk brush, the tassel bag is then fastened in place over the pollinated shoot to protect the developing ear.

Genetic resources for crop improvement include the wild relatives of a particular crop. Maize was the staple grain upon which pre-Columbian civilizations in the Americas were founded. Its wild relatives include six species of wild Zea, common name teosinte, that are endemic to Mexico and Guatemala west of the Sierra Madre Oriental mountain range, and twelve to sixteen species of Tripsacum, common name gamagrass, that range throughout North and South America from Canada to Chile.

The annual teosintes (Z. mays ssp. mexicana, Z. m. ssp. parviglumis, Z. huehuetenangensis, Z. luxurians and a perennial (Z. diploperennis) all have the same chromosome number as maize (n=10) and are cross-fertile with maize. The sixth species Z. perennis is a tetraploid perennial whose chromosome number is 2n=40. Normal diploid maize is not cross-fertile with the tetraploid perennial teosinte. However, cross-fertility can be achieved by treating maize to double its chromosome number to 2n=40, which can be crossed with Z. perennis to produce fertile 2n=40 hybrids (Shaver 1964). When fifty per cent maize segregates selected from the 2n=40 hybrids, 2n=30 triploid plants that were also fertile were recovered. However, all attempts to derive diploid hybrids between maize and Z. perennis failed to produce fertile offspring.

Tripsacum is a polyploid, rhizomatous perennial grass more distantly related to maize that has a different chromosome number (x=18). Tripsacum species are highly variable in form, vigor, and ecological preference. Adaptations range from seasonally swampy sites, to sandy soils, to tropical habitats and to near-desert conditions. Tripsacum, which is not known to form fertile hybrids with maize or with Zea naturally, has valuable agronomic characters that could be exploited for the overall improvement of maize but is hindered by the problem of cross sterility (Kindiger and Beckett 1990).

The progeny of (maize X Tripsacum) obtained by artificial methods have ten maize chromosomes and either 18 or 36 Tripsacum chromosomes and are male sterile. Female fertility can be partially restored using special techniques that eliminate most of the Tripsacum chromosomes (Mangelsdorf 1974). Plants obtained by crossing Tripsacum and maize (Zea mays L.) employing Tripsacum as the pollen donor have unreduced gametes with a complete set of Zea chromosomes and a complete set of Tripsacum chromosomes. There is one report of a successful reciprocal cross in which Tripsacum was pollinated by maize that required a special culture technique to bring the embryos to maturity, but the plants were sterile (Farquharson 1957). Maize-Tripsacum hybrids have been crossed with teosinte to created a trigenomic hybrid that has a total of 38 chromosomes; 10 from maize, 18 from Tripsacum and 10 from teosinte. The resulting trigenomic plants were all male sterile with a high degree of female infertility (Mangelsdorf 1974; Galinat 1986).

Based on known crossability relationships between Zea and Tripsacum and the results of prior crosses between them, the success of the crosses between teosinte and Tripsacum resulting in viable, fully fertile plants with chromosome numbers of 2n=20 (Eubanks 1995, 1997) could not have been predicted. Reduction in chromosome number in the interspecific crosses was unexpected based on prior art. The fertility of plants resulting from the cross made both ways with Tripsacum as pollen donor and pollen recipient was also unexpected based on prior art.

Although the base chromosome numbers of Tripsacum and teosinte are different, x=10 in Zea and x=18 in Tripsacum, the respective total chromosome lengths of Tripsacum and diploid perennial teosinte are almost equal. The total length of the 18 Tripsacum dactyloides chromosomes is 492.5μ (Chandravadana et al. 1971), and the total length of the 10 Zea diploperennis chromosomes is 501.64μ (Pasupuleti and Galinat 1982). It is not easy to obtain a hybrid plant when crossing Tripsacum and teosinte. Hundreds of pollinations are required to obtain a viable seed, and approximately half of seedlings that germinate die soon after germination. However, as evidenced by cross fertility and chromosome number, when precise alignments occur between homologous regions of the chromosomes of Tripsacum and teosinte there is a sufficient degree of pairing to occasionally enable the rare and unexpected success of this cross.

The unexpected fertility of Tripsacum-teosinte hybrids, and their cross-fertility with maize, are of great value because they provide opportunity for directly crossing the chimeric recombinants with maize. Tripsacum-teosinte hybrids provide a genetic bridge for importing new Tripsacum genes not found in maize or the wild Zeas, as well as novel genetic material formed in the genomic reorganization between the two species that gives rise to viable, fertile plants that can be crossed with maize using traditional plant breeding techniques.

DNA fingerprinting has revealed that new Tripsacum alleles not found in maize or the wild Zeas and new recombinant DNA fragments not found in either parent are stably inherited in the progeny of succeeding generations and in crosses with maize. The novel DNA fragments and alleles unique to Tripsacum are stably inherited in succeeding generations of maize X Tripsacum-teosinte. For purposes herein, unique genetic material refers to regions where new DNA fragments are repeatedly and reliably formed whenever crosses between Tripsacum and teosinte produce viable, fertile plants.

Feasibility has been demonstrated in plants derived from crossing Tripsacum-teosinte recombinants with maize that are resistant to corn rootworm (Diabrotica sp.) and corn borer, are drought tolerant, have properties of perennialism, develop aerenchyma tissue in their roots, and can grow in low pH conditions. Investigation and characterization of other improvements to maize including herbicide tolerance, aflatoxin resistance, and enhanced grain quality are underway.

References Cited

U.S. PATENT DOCUMENTS

| | | |
|---|---|---|
| 4,545,146 | 10/1985 | Davis |
| 4,627,192 | 12/1986 | Fick |
| 4,684,612 | 8/1989 | Hemphill et al |
| 4,737,596 | 4/1988 | Seifert et al. |
| 4,837,152 | 6/1989 | Hemphill and Warshaw |
| 5,059,745 | 10/1991 | Foley |
| PP 6,906 | 7/1989 | Eubanks |
| PP 7,977 | 9/1992 | Eubanks |
| 5,330,547 | 7/1994 | Eubanks |
| PP 9,640 | 9/1996 | Eubanks |
| 5,750,828 | 5/1998 | Eubanks |

Other Publications

Armstrong, W. 1979. Aeration in higher plants. Pp. 226-332 in Advances in Botanical Research, H. W. Woolhouse, ed. Academic Press, New York.

Chaganti, R. S. K. 1965. Cytogenetic studies of maize-Tripsacum hybrids and their derivatives. Harvard Univ. Bussey Inst., Cambridge, Mass.

Chandravadana, P., W. C. Galinat and B. G. S. Rao. 1971. A cytological study of Tripsacum dactyloides. J. Heredity 62:280-284.

Chomet, P. S. 1994. Transposon tagging with Mutator. In M. Freeling and V. Walbot (eds.), The Maize Handbook, Springer-Verlag, New York.

Clark, R. B., E. E. Alberts, R. W. Zobel, T. R. Sinclair, M. S. Miller, W. D. Kemper and C. D. Foy. 1996. Eastern gamagrass (Tripsacum dactyloides) root penetration and chemical properties of claypan soils. Pp. 191-211 in Root Demographics and Their Efficiencies in Sustainable Agriculture, Grasslands and Forest Ecosystems. James E. Box, Jr., ed. Kluwer Academic Publishers, Dordrecht, The Netherlands.

Cohen, J. I. and W. C. Galinat. 1984. Potential use of alien germplasm for maize improvement. Crop Science 24:1011-1015.

Comis, Don. 1997. Aerenchyma: lifelines for living underwater. Agricultural Research 45:4-8.

Curtis, H. and N. S. Barnes. 1989. Biology. Worth Publishers, Inc. New York, N.Y.

Drew, M. C. and L. H. Stolzy. 1996. Growth under oxygen stress. Pp. 397-414 in Plant Roots: The Hidden Half, Y. Waisel, A. Eshel and U. Kafkafi, eds. Marcel Dekker, Inc., New York.

Esau, K. 1977. Anatomy of Seed Plants. John Wiley & Sons, New York.

Eubanks, M. W. 1995. A cross between two maize relatives: Tripsacum dactyloides and Zea diploperennis (Poaceae). Economic Botany 49:172-182.

Eubanks, M. W. 1997. Molecular analysis of crosses between Tripsacum dactyloides and Zea diploperennis (Poaceae). Theor. Appl. Genet. 94:707-712.

Eubanks, M. W. 2002, Investigation of Novel Genetic Resource for Rootworm Resistance in Corn. Pp. 2544-2554 in Proceedings of the 2002 National Science Foundation Design, Service and Manufacturing Grantees and Research Conference, Jan. 7-10, 2002, San Juan, Puerto Rico. CD ROM produced by the College of Engineering, Iowa State University, Ames, Iowa.

Farquharson, L. I. 1957. Hybridization of Tripsacum and Zea. J. Heredity 48:295-299.

Foy. C. D. 1996. Eastern gamagrass (Tripsacum dactyloides) root penetration and chemical properties of claypan soils. 5th Symposium of the International Society of Root Research, July 14-18, Clemson, S.C.

Foy, C. D. 1997. Tolerance of Eastern gamagrass to excess aluminum in acid soil and nutrient solution. Journal of Plant Nutrition 20: 1119-1136.

Galinat, W. C. 1974. Intergenomic mapping of maize, teosinte and Tripsacum. Evolution 27:644-655.

Galinat, W. C. 1977. The origin of corn. In G. F. Sprague (ed.). Corn and Corn Improvement. Amer. Soc. Agronomy, Madison, Wis.

Galinat, W. C. 1982. Maize breeding and its raw material. In W. L. Sheridan (ed.) Maize for Biological Research. University Press, Grand Forks, N.D.

Galinat, W. C. 1986. The cytology of the trigenomic hybrid. Maize Genetics Newsletter 60:133.

Gardiner, J. E. H. Coe, S. Melia-Hancock, D. A. Hoisington and S. Chao. 1993. Development of a core RFLP map in maize using an immortalized F₂ population. Genetics 134:917-930.

Griffiths, A. J. F., J. H. Miller, D. T. Susuki, R. C. Lewin, and W. M. Gelbart. 1993. An Introduction to Genetic Analysis, 5th edition. W. H. Freeman and Co., New York.

Griliches, Zvi. 1957. Hybrid corn: an exploration in the economics of technological change. Econometrica 25:501-519.

Helentjaris, T., M. Slocum, S. Wright, A. Schaefer and J. Nienhuis. 1986. Construction of genetic linkage maps in maize and tomato using restriction fragment length polymorphisms. Theor. Appl. Genet. 72:761-769.

Jeffreys, A. J., N. J. Royle, W. Wilson and Z. Wong. 1988. Spontaneous mutation rates to new length alleles at tandem-repetitive hypervariable loci in human DNA. Nature 332:278-281.

Justin, S. and W. Armstrong. 1987. The anatomical characteristics of roots and plant response to soil flooding. New Phytologist 106:465-495.

Kindiger, B. and J. B. Beckett. 1990. Cytological evidence supporting a procedure for directing and enhancing pairing between maize and Tripsacum. Genome 33:495-500.

Kresovich, S., W. F. Lamboy, A. K. Szewc-McFadden, J. R. McFerson, and P. L. Forsline. 1993. Molecular diagnostics and plant genetic resources conservation. Agbiotech News and Information 5(7) :255-258.

Lewin, B. 1997. Genes V. Oxford University Press, Oxford, UK.

Liu, S., R. G. Cantrell, J. C. McCarty, Jr. and J. McD. Stewart. 2000. Simple sequence repeat-based assessment of genetic diversity in cotton race stock accessions. Crop Science 40:1459-1469.

Maguire, M. P. 1961. Divergence in Tripsacum and Zea chromosomes. Evolution 15:393-400.

Maguire, M. P. 1963. Chromatid interchange in allodiploid maize-Tripsacum hybrids. Can. J. Genet. Cytol. 5:414-420.

Mangelsdorf, P. C. 1974. Corn: Its Origin, Evolution and Improvement. Harvard Univ. Press, Cambridge, Mass.

Mangelsdorf, P. C., and R. G. Reeves. 1931. Hybridization of maize, Tripsacum and Euchlaena. Journal of Heredity 22:329-343.

Melchinger, A. E., M. M. Messmer, M. Lee, W. L. Woodman, and K. R. Lamkey. 1991. Diversity and relationships among U.S. maize inbreds revealed by restriction fragment length polymorphisms. Crop Science 31:669-678.

Messmer, M. M., A. E. Melchinger, R. Herrmann, and J. Boppenmaier. 1993. Relationships among early European maize inbreds: II. Comparison of pedigree and RFLP data. Crop Science 33:944-950.

Morris, M. L., Ed. 1998. Maize Seed Industries in Developing Countries. Lynne Rienner Publishers, Inc., Boulder, Colo.

Neuffer, M. G. 1982. Growing maize for genetic purposes. In W. L. Sheridan (ed.) Maize for Biological Research. University Press, Grand Forks, N.D.

Neuffer, M. G., E. H. Coe and S. R. Wessler. 1997. Mutants of Maize. Cold Spring Harbor Laboratory Press, New York.

Pasupuleti, C. V. and W. C. Galinat. 1982. Zea diploperennis I. Its chromosomes and comparative cytology. Heredity 73:168-170.

Poehlman, J. M. 1986. Breeding Field Crops. 3rd ed. AVI Publ. Co., Inc., Westport, Conn.

Ray, J. D., B. Kindiger and T. R. Sinclair. 1999. Introgressing root aerenchyma into maize. Maydica 44:113-117.

Reeves, R. G. and A. J. Bockholt. 1964. Modification and improvement of a maize inbred by crossing it with Tripsacum. Crop Science 4:7-10.

Senior, M. L., J. P. Murphy, M. M. Goodman, and C. W. Stuber. 1998. Utility of SSRs for determining genetic similarities and relationships in maize using an agarose gel system. Crop Science 38:1088-1098.

Shaver, D. L. 1964. Perennialism in Zea. Genetics 50:393-406.

Smith, J. S. C., E. S. L. Chin, H. Shu, O. S. Smith, S. J. Wall, M. L. Senior, S. E. Mitchell, S. Kresovitch, and J. Ziegle. 1997. An evaluation of the utility of SSR loci as molecular markers in maize (Zea mays L.): Comparisons with data from RFLPs and pedigree. Theoretical and Applied Genetics 95:163-173.

Smith, O. S. and J. S. C. Smith. 1992. Measurement of genetic diversity among maize hybrids; a comparison of isozymic, RFLP, pedigree, and heterosis data. Maydica 37:53-60.

Stadler, L. J. 1951. Spontaneous mutation in maize. Cold Spring Harbor Symp. Quant. Biol. 16:49-63.

Tantravahi, 1968. Cytology and crossability relationships of Tripsacum. Harvard Univ. Bussey Inst., Cambridge, Mass.

### SUMMARY OF THE INVENTION

In one embodiment of the invention, there is provided a method for screening a plant to determine whether said plant is a cross between Tripsacum and teosinte. In the steps of the method, the total genomic DNA is isolated from the plant; then the genomic DNA is digested with one to five restriction enzymes from the group consisting of EcoRI, EcoRV, HindIII, BamHI and MspI; then the restriction digested DNA is probed with one or more DNA markers selected from the group consisting of the maize nuclear DNA probes, maize mitochondrial DNA probes, and Tripsacum DNA probes listed in Table 1; then determining if one or more of the novel recombinant chimeric restriction fragments characterized by the respective marker-restriction enzyme association and fragment sizes listed in Table 2 is present, or if one or more of the introgressed Tripsacum fragments characterized by the respective marker-restriction enzyme association and fragment sizes listed in Table 3 is present. To produce a Tripsacum-teosinte recombinant plant, a Tripsacum plant is pollinated by pollen from a teosinte plant by controlled pollination technique, or reciprocally, a teosinte plant is pollinated by pollen from a Tripsacum plant. The resulting intergeneric hybrids are fully fertile and cross-fertile with maize. The hybrid plants are characterized by their utility as a genetic bridge to transfer novel genetic materials into maize and their unexpected chromosome number of 2n=20 instead of 2n=28 or 2n=46. This invention relates to hybrid seed, hybrid plants produced by the seed and/or tissue culture, variants, mutants, modifications, and cellular and molecular components of the hybrid plants that contain novel genetic materials derived from (Tripsacum X teosinte) or (teosinte X Tripsacum).

In another embodiment of the invention, there is provided a method for screening a plant to determine whether said plant is a cross between maize and a Tripsacum-teosinte hybrid plant. In the steps of the method, the total genomic DNA is isolated from the plant; then the genomic DNA is digested with one to five restriction enzymes from the group consisting of EcoRI, EcoRV, HindIII, BamHI and MspI; then the restriction digested DNA is probed with one or more DNA markers selected from the group consisting of the maize nuclear DNA probes, maize mitochondrial DNA probes, and Tripsacum DNA probes listed in Table 1; then determining if one or more of the novel recombinant chimeric restriction fragments characterized by the respective marker-restriction enzyme association and fragment sizes listed in Table 2 is present, or if one or more of the introgressed Tripsacum fragments characterized by the respective marker-restriction enzyme association and fragment sizes listed in Table 3 is present. To produce a maize X Tripsacum-teosinte plant or the reciprocal Tripsacum-teosinte X maize plant, the intergeneric hybrid plant (Tripsacum X teosinte) or (perennial teosinte X Tripsacum) is crossed with maize by controlled pollination. In the cross, the pollen of (Tripsacum X teosinte) or (teosinte X Tripsacum) is transferred to maize silks, or maize pollen is transferred to the silks of (Tripsacum X teosinte) or (teosinte X Tripsacum). This invention relates to hybrid seed, hybrid plants produced by the seed and/or tissue culture, variants, mutants, modifications, and cellular and molecular components of the hybrid plants that contain novel genetic materials derived from maize X (Tripsacum X teosinte) or maize X (teosinte X Tripsacum).

In another embodiment of the invention, there is provided a method for screening a maize plant to determine whether said plant is a backcross between maize and a (maize X Tripsacum-teosinte) hybrid plant. In the steps of the method, the total genomic DNA is isolated from the plant; then the genomic DNA is digested with one to five restriction enzymes from the group consisting of EcoRI, EcoRV, HindIII, BamHI and MspI; then the restriction digested DNA is probed with one or more DNA markers selected from the group consisting of the maize nuclear DNA probes, maize mitochondrial DNA probes, and Tripsacum DNA probes listed in Table 1; then determining if one or more of the novel recombinant chimeric restriction fragments characterized by the respective marker-restriction enzyme association and fragment sizes listed in Table 2 is present, or if one or more of the introgressed Tripsacum fragments characterized by the respective marker-restriction enzyme association and fragment sizes listed in Table 3 is present. To produce a backcross hybrid maize plant, the hybrid plant obtained from maize X (Tripsacum X teosinte) or maize X (teosinte X Tripsacum) is backcrossed to maize. In the backcross, the pollen of the trigeneric hybrid plant is transferred to the silks of one of the original parents (Tripsacum X teosinte) or (teosinte X Tripsacum) or maize. This invention relates to hybrid seed, hybrid plants produced by the seed and/or tissue culture, variants, mutants, modifications, and cellular and molecular components of the hybrid plants that contain novel genetic materials derived from [maize X (Tripsacum X teosinte)] maize or maize X [maize X (teosinte X Tripsacum)].

In another embodiment of the invention, there is provided plants and plant tissues produced by the method of crossing maize with a Tripsacum-teosinte hybrid that contain novel genetic materials and exhibit beneficial agronomic traits. For example, these plants may contain novel genes for such traits as pest and pathogen resistance, drought tolerance, cold tolerance, water-logging tolerance, improved grain quality, improved forage quality, totipotency, perennialism, tolerance to acidic soils, tolerance to high-aluminum soils, herbicide tolerance, tolerance to toxic metals, enhanced adaptability in a carbon dioxide enriched environment, roots with aerenchyma, and ability to attract nitrogen-fixing bacteria to the rhizosphere. These plants can be employed in recurrent selection breeding programs to select for maize inbred and hybrid lines that exhibit such traits.

For the purposes of this application, the following terms are defined to provide a clear and consistent description of the invention.

Aerenchyma. Formation of large intercellular spaces in the root cortex, i.e. the ground tissue region between the vascular tissue and the epidermis.

Allele. One of the different forms of a gene that can exist at a single locus.

Autoradioaraphy. A process in which radioactive materials are incorporated into cellular components, then placed next to a film or photographic emulsion to produce patterns on the film that correspond to the location of the radioactive compounds within the cell.

Electrophoresis. A technique for separating the components of a mixture of molecules (proteins, DNAs, or RNAs) in an electric field within a gel matrix.

Genetic markers. Alleles used as experimental probes to keep track of an individual, a tissue, a cell, a nucleus, a chromosome, or a gene.

Gene. The fundamental physical and functional unit of heredity that carries information from one generation to the next. The plant gene is “a DNA sequence of which a segment is regularly or conditionally transcribed at some time in either or both generations of the plant. The DNA is understood to include not only the exons and introns of the structural gene but the cis 5′ and 3′ regions in which a sequence change can affect gene expression” (Neuffer, Coe and Wessler 1997).

Genotype. The allelic composition of a cell—either of the entire cell or, more commonly, for a certain gene or a set of genes of an individual.

Hybrid plant. An individual plant produced by crossing two parents of different genotypes or germplasm backgrounds.

Inbred. A plant that has been self pollinated or sib mated.

Inversion. A chromosomal aberration in which the order of a chromosomal segment has been reversed.

Linkage group. A group of genes that have their loci on the same chromosome.

Locus. The place on a chromosome where a gene is located.

Molecular genetics. The study of the molecular processes underlying gene structure and function.

Mutagen. An agent that is capable of increasing the mutation rate.

Mutation. (1) The process that produces nucleotide sequences, genes, genetic elements, or chromosomes differing from the wild-type. (2) The nucleotide sequences, genes, genetic elements, or chromosomes that result from such a process.

Phenotype. The observable properties of an organism that are genetically controlled.

Plant breeding. The application of genetic analysis to development of plant lines better suited for human purposes.

Polymorphism. The existence of two or more distinct, segregating forms in a population.

Probe. Defined nucleic acid segment that can be used to identify specific molecules bearing the complementary DNA or RNA sequence, usually through autoradiography.

Restriction enzyme. An endonuclease that will recognize specific target nucleotide sequences in DNA and cut the DNA at these points; a variety of these enzymes are known and used extensively used in genetic engineering and molecular biology.

RFLP. Refers to restriction fragment length polymorphism of a specific size determined by its molecular weight in kilobases that is visualized on a Southern blot when a radiolabelled DNA probe of a specific sequence of known bases hybridizes to the fragment that contains that particular DNA sequence. RFLPs are considered to represent an allele of a gene. When they have been mapped to precise chromosomal loci as in maize, they provide a highly reliable fingerprinting method for precision genotyping of individuals.

Robertsonian fusion. A chromosomal aberration that involved the fusion of long arms of acrocentric chromosomes at the centromere.

SSRs. Simple sequence repeat polymorphisms are intergenic tandem repeats of 2 to 6 base pairs that are amplified by polymerase chain reaction (PCR) using primers complimentary to the flanking regions of the repeats. The PCR products are separated by electrophoresis, and the codominant polymorphisms are visualized as different bands on the gel. SSR variability can be scored as accurately and reliably as RFLP polymorphisms. SSRs are rapidly becoming the molecular markers of choice for genotyping, as well as for identifying and mapping genes and assessing genetic diversity.

Southern blot. Transfer of electrophoretically separated fragments of DNA from the gel to an absorbent surface such as paper or a membrane which is then immersed in a solution containing a labeled probe that will bind to homologous DNA sequences.

Totipotency. The ability of a cell to proceed through all the stages of development and thus produce a normal adult.

Translocation. A chromosomal mutation associated with the transfer of a chromosomal segment from one chromosome to another.

Wild type—refers to a reference and it can mean an organism, set of genes, gene or nucleotide sequence. For purposes herein the wild type refers to the parents of hybrid progeny.

### DESCRIPTION OF THE DRAWING

FIG. 1 is a schematic drawing of the 10 linkage groups of maize. The open circles represent approximate positions of the centromeres. The relative positions of the RFLP marker probes that were used to DNA fingerprint recombinant plants derived from crossing Tripsacum and teosinte, plus plants derived from crossing maize with Tripsacum-teosinte recombinants, are indicated on each of the ten maize chromosomes. Molecular markers at loci where stable, heritable variant fragments that are not found in either parent are underscored, and markers that indicate where new alleles from a Tripsacum parent that are not found in Zea have been inherited in the Tripsacum-teosinte recombinant progeny and in crosses between maize and Tripsacum-teosinte are italicized.

FIG. 2 is a schematic drawing of the 10 linkage groups of maize. The open circles represent approximate positions of the centromeres. Corresponding SSR marker probes are listed beneath the RFLP marker probes used to DNA fingerprint recombinant plants derived from crossing Tripsacum and teosinte, plus plants derived from crossing maize with Tripsacum-teosinte recombinants.

### DETAILED DESCRIPTION OF THE INVENTION

The principles and techniques that were used to detect the chimeric genetic material and novel Tripsacum alleles are commonly used to fingerprint crop varieties (Kresovich et al. 1993). First DNA is extracted and isolated from plant samples; then the DNA is cut into fragments using restriction enzymes that cut at precise nucleotide sequences; the fragments are then separated by size, i.e. molecular weight, on an agarose gel by electrophoresis; the DNA is then denatured, i.e. separated into single strands, and transferred to a filter or membrane that binds single-stranded but not double-stranded DNA, a method referred to as Southern blotting. The restriction fragments are immobilized on the filter in the same way they are positioned on the electrophoretic gel. The membrane is then incubated in a solution containing multiple copies of a radiolabeled probe for a particular DNA sequence that has been mapped to a certain chromosomal locus or loci in the maize genome. The probe hybridizes to homologous DNA sequences to reveal the distinctive bands of specific molecular weight sizes that are formed by a particular restriction enzyme/probe combination in any individual plant. The bands, i.e. the restriction fragment length polymorphisms, are then visualized in the resulting autoradiograph. Like a bar code which the RFLP bands resemble, they precisely identify the genotype of individual plants. This method of RFLP genotyping provides information necessary to distinguish between plants whose genetic composition may differ only slightly. This DNA fingerprinting technique permits the unambiguous identification of genotypes (Melchinger et al. 1991; Messmer et al. 1993). Fingerprinting profiles are routinely used for genetic identity analysis to classify closely related materials, estimate genetic distances, determine paternity, and complement conventional pedigree records in commercial hybrid production (Smith and Smith 1992).

Although maize contains many duplicate genes, it is generally thought of as a diploid organism in which the progeny of maize hybrids inherit one allele for a trait from one parent and another allele for that trait from the other parent. In the DNA fingerprint of a single gene that is not duplicated elsewhere in the genome and the offspring inherit the same polymorphism marked by a molecular probe that maps to the specific region of the particular chromosome to which the trait being investigated has been mapped from both parents, they will be homozygous for that particular trait and a single band will be seen on the autoradiograph. If the progeny inherit different polymorphisms from each parent plant, they will be heterozygous at that locus and two bands will be detected on the autoradiograph, one band from one parent and a different band from the other parent. Multiple bands are seen at more complex loci involving gene duplication. In general, the offspring of two parents can be identified by comparing their DNA fingerprints to those of the parents because progeny exhibit a combination of bands from both parents. Sometimes, however, the progeny of known parentage exhibit a band or bands that are not found in either parent. Such novel bands may arise from mutations in the nucleotide sequences or from chromosomal mutations that cause genomic reorganization such that some RFLP bands will be different from both of parents (Griffiths et al. 1993).

Such mutant or novel rearrangements in the genetic material are revealed by comparative analysis of the RFLP bands of the parent plants and hybrid progeny. Bands present in the offspring not found in either parent indicate regions of the genome where novel genetic material has arisen, i.e. mutations have occurred. As stated above, mutations are rare, and in most cases deleterious. Broadly speaking among all organisms, mutation rates vary and they range from 1 in 1,000 to 1 in 1,000,000 gametes per generation depending on the gene involved (Curtis and Barnes 1989). For example, each human with approximately 100,000 genes is expected to carry 2 mutant alleles. The unique restriction fragments of the Tripsacum-teosinte hybrids occur at 148 out of 176 loci and are unprecedented in their high mutation rate. Furthermore, the novel polymorphisms are stably inherited in succeeding generations of Tripsacum-teosinte progeny and of maize X Tripsacum-teosinte progeny. In addition to the rarity and usual deleterious effect of mutations, a basic biological tenet is that mutations occur at random or by chance (Lewin 1997). In a study of spontaneous mutation rates to new length alleles at tandemly repeated loci in human DNA (Jeffreys et al. 1988) mutations arose sporadically and there was no clustering of mutations within a family. Siblings never shared a common mutant allele. Therefore, it is unexpected that the same mutations would recur not only among siblings but among hybrids of different parentage. Thus it is remarkable and unexpected that the same unique polymorphisms are repeatedly found in hybrid progeny derived from crossing different Tripsacum and different teosinte parent plants (see Table 2), and that those same novel restriction fragments are stably inherited in crosses between Tripsacum-teosinte hybrids and maize (see Table 2). These unique RFLPs provide a rich new source of variant genetic material for selection in corn improvement.

In molecular assays performed by Linkage Genetics, Salt Lake City, Utah, and Biogenetics, Inc., Brookings, S.D., DNA was isolated from different F₁, F₂ and F₃ hybrids between Tripsacum and teosinte, the parents of these hybrids, W64A and B73 maize inbred lines, as well as F₁, F₂, F₃ and F₄ hybrids between maize and Tripsacum-teosinte. The protocol for DNA isolation, restriction enzyme digestion, Southern blotting, probe hybridization, and analysis of autoradiographs has been described by Helentjaris et al. (1986). Internal standards of known molecular weights and a ladder were included in the gels to characterize molecular weights of the bands and facilitate scoring accuracy and analysis.

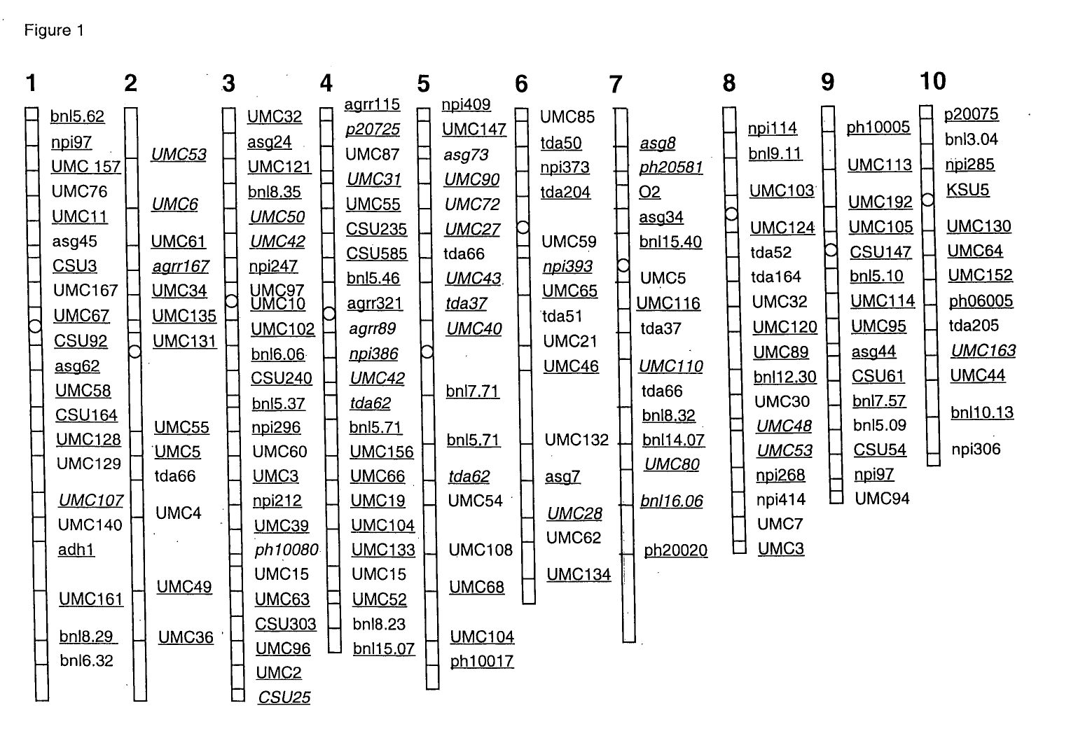

Total genomic DNA from the individual parent and hybrid plants was digested with from at least one of five different restriction enzymes, EcoRI, EcoRV, HindIII, BamHI, and MspI, then transferred to Southern blots, and probed with 176 publicly available DNA markers which include a majority of maize nuclear DNA probes mapped to the ten linkage groups of maize (Gardiner et al. 1993), six maize mitochondrial probes, and some Tripsacum (tda) probes for which the loci have not yet been mapped to the maize genome. The molecular markers on the genetic linkage map of maize were mapped by recombinational analyses based on proof of the identity of a clone. Thus each locus represents a gene based on clone identification (Neuffer, Coe and Wessler 1997). The 176 molecular markers that were employed in DNA fingerprinting of parent species, Tripsacum-teosinte hybrids, and (maize X Tripsacum-teosinte) are listed in Table 1. FIG. 1 depicts the orders and approximate locations of the mapped probes on the ten maize chromosomes (cf. Neuffer, Coe and Wessler 1997). A large number of the probes reveal bands that are not present in either parent of a particular progeny. These novel bands signal loci where mutations occurred in the process of intergeneric hybridization. Their approximate mapped loci on the ten chromosomes of Zea are shown in FIG. 1, and they are indicated in Table 1 by underscoring. There are also loci where Tripsacum polymorphisms are present in Tripsacum-teosinte hybrids that were not present in the genotyped maize lines and other teosinte species. These unique Tripsacum polymorphisms can be used to screen for introgression of Tripsacum alleles in maize via the Tripsacum-teosinte genetic bridge. They are italicized in Table 1 and FIG. 1.

Crosses have been made using seven different Tripsacums including three accessions of Tripsacum dactyloides, one from Santa Claus, Ind. (4n=72), one from Hilltop Experiment Station, Bloomington, Ind. (4n=72), and one from Manhattan, Kan. (2n=36); Tripsacum laxum (CEL 48770)from Veracruz, Mexico, Tripsacum peruvianum (DHT-66-13-01) from San Martin, Peru, Tripsacum manisurioides 37553 from Woodward, Okla., Tripsacum floridanum MIA34719 from Florida, and Tripsacum sp. from Nabogame, Sonora, Mexico. The Tripsacums have been crossed with teosinte plants of Zea diploperennis originating from different populations in Jalisco, Mexico; plants 3-7 and 3-3 from a population in Upper las Joyas, Sierra de Manantlan, Iltis, Nee and Guzman accession number 1250, January 1979, and plant 2-4 from a La Ventana population, R. Guzman Accession number 777, Dec. 14, 1977, and with (maize X Tripsacum-teosinte) hybrids. The Tripsacum-teosinte hybrids included in Tables 2 and 3 are: (1) Sun Dance, Zea diploperennis 3-7 X Tripsacum dactyloides (2n=72); (2) Tripsacorn, Tripsacum dactyloides (2n=72) X Zea diploperennis 3-3; (3) Sun Star, Zea diploperennis 2-4 X Tripsacum dactyloides (2n=36); (4) Sun Devil, Tripsacum dactyloides (2n=72) X Zea diploperennis; (5) 20A, Zea diploperennis 2-4 X Tripsacum dactyloides (2n=72). There have been multiple crosses between the maize inbred lines W64A, B73 and A188 with various Tripsacum-teosinte hybrids. Hybrids between Tripsacum-teosinte and maize included in Tables 2 and 3 are: 64SS (W64A X Sun Star), 64TC (W64A X Tripsacorn), 2019 (B73 X Tripsacorn), 4021 (B73 X Tripsacorn), 3024 (B73 X Tripsacorn), 3028 (B73 X Tripsacorn backcrossed to Tripsacorn), 3125 (W64A X Tripsacorn), 4126 (W64A X Tripsacorn), 3029 (B73 X Tripsacorn), 4029 (B73 X Tripsacorn), 10 individuals of TC64 (Tripsacorn X W64A), 7022 (TC64 backcrossed to Tripsacorn), 7024 (Tripsacorn X W64A), 9094 X 7009 (an advanced maize line in a B73/W64A maize background introgressed with Tripsacorn and Sun Star), 97-5 X 97-1 (an advanced maize line in a B73/W64A maize background introgressed with Tripsacorn and Sun Star), and V70 (an advanced maize line in a W64A/A188 maize background introgressed with Tripsacorn and Sun Star). Other hybrids include 20B, Zea diploperennis 2-4 X Tripsacum dactyloides (2n=72); Devil Corn, a cross between Sun Devil and Tripsacorn; [(7022 X Devil Corn) X Tripsacum laxum]; 7022 X Tripsacum manisurioides; TC64#5 X Nabogame Tripsacum sp.; TC64#5 X Tripsacum floridanum, and (7022 X Devil Corn) X Tripsacum peruvianum.

Tables 2 identifies the molecular marker loci associated with novel restriction fragments, indicates their molecular weight, and specifies in which Tripsacum-teosinte hybrids and [maize X (Tripsacum-teosinte)] lines they occur. Table 3 identifies the molecular markers associated with unique Tripsacum RFLPs, indicates their molecular weight, and specifies their inheritance in the Tripsacum-teosinte hybrids plus exemplary (maize X Tripsacum-teosinte) lines in which they are found.

In order to determine which Tripsacum polymorhphisms are present in Tripsacum-teosinte hybrids that are not present in other Zeas, 5 to 13 individuals from populations of two modern maize inbred lines, B73 and W64A, four indigenous Latin American maize races, Nal Tel (Yuc7), Chapalote (Sin), Pollo (Col 35 ICA), and Pira (PI44512), and the six wild Zeas, Z. mexicana (PI566683 and PI566688), Z. parviglumis (PI384061 and PI331785), Z. luxurians (PI306615), Z. huehuetenangensis (Ames21880), Z. diploperennis and Z. perennis (Ames 21875), were DNA fingerprinted with the probes in Table 1 and FIG. 1. The molecular marker loci are identified by the specific probe/restriction enzyme combination and molecular weight. Table 4 gives the molecular weights of parental RFLPs for comparative reference.

The novel genetic materials, which include the new restriction fragments formed in the wide cross genomic reorganization and unique polymorphisms from Tripsacum not found in maize or the wild Zeas, have been shown to be stably inherited in three generations of Tripsacum-teosinte hybrids, and eight generations of Tripsacum-teosinte hybrids that were crossed with maize. The unique Tripsacum polymorphisms and recombinant chimeric RFLPS, their heritability in succeeding generations of Tripsacum-teosinte hybrids, and their transmissibility to maize is unprecedented and unexpected based on prior art. These novel DNA fragments have utility for genetic analysis of Zea, and selection of new variant alleles that may enhance traits such as insect and disease resistance, drought stress tolerance, cold tolerance, herbicide tolerance, perennialism, increased grain yield, totipotency, apomixis, better root systems, tolerance of water-logged soils, tolerance of high-aluminum and acidic soils, improved grain quality, and improved forage quality. When these novel RFLPs co-segregate with crop improvement traits, they can be successfully employed in recurrent selection breeding programs for early and rapid screening of plants carrying the desired trait. They are also important for identifying the regions of the genome where the genes for the trait reside.

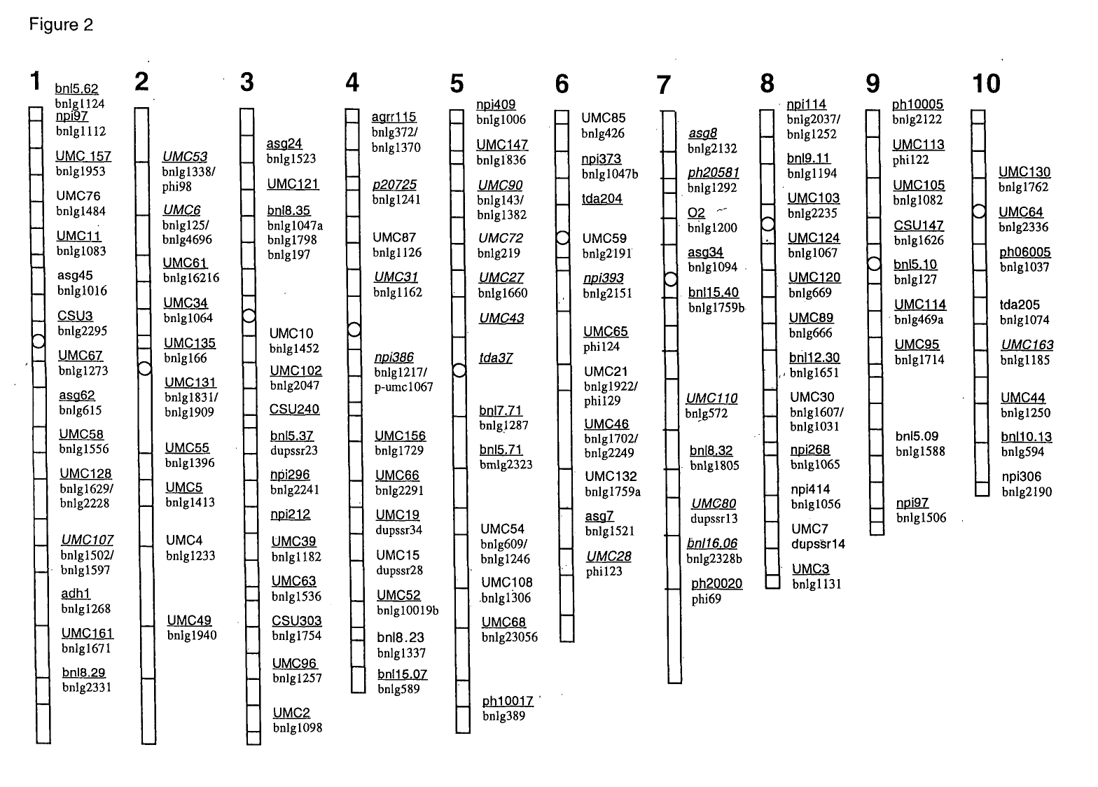

Examples of the application of these molecular markers for genetic analysis and marker-assisted breeding are described in regard to identification of marker loci associated with two traits that are characteristic of Tripsacum and have been transferred into maize via the Tripsacum-teosinte bridging cross. They include resistance to the insect pest corn rootworm (Diabrotic virgifera Le Conte), and formation of aerenchyma in the roots. Aerenchyma tissue consists of large spaces in the root cortex that allow movement of oxygen from the aboveground plant tissue to the roots, an adaptation to anaerobic environments (Comis 1997). Aerenchyma allow the roots to penetrate deep in the soil below the hard pan which greatly enhances drought tolerance. It allows the plant to survive in saturated soils. Genomic DNA isolated from leaves of Tripsacum-teosinte hybrid plants and Tripsacum-teosinte X maize hybrid plants that demonstrated resistance to corn rootworm in insect bioassays was subjected to RFLP genotyping as described above. In Table 2 the Tripsacum-teosinte hybrids that exhibit rootworm resistance are Tripsacorn, Sun Star and 20A, and the Tripsacum-teosinte X maize plants that were resistant to corn rootworm are 2019, 3024, 3028, 3125, 4126 and TC64. The fact that the Tripsacum-teosinte hybrid called Sun Dance is not resistant provides a unique opportunity to simplify genetic analysis and determine the molecular markers and chromosomal regions to which this trait may be assigned without having to map a large segregating population. This can be done by examining all the unique polymorphisms in Tables 2 and 3 and identifying which ones are found only in Tripsacorn, Sun Star, 20A, 2019, 3024, 3028, 3125, 4126 and TC64. Since only one molecular marker satisfies this requirement, UMC103 on the short arm of chromosome 8, it is clearly a marker for rootworm resistance. However, since the trait is not expressed in a 3:1 ratio according to simple Mendelian inheritance, and the trait is either expressed in lower frequencies than expected, or expression may be lost in subsequent generations, more than one loci are affecting expression. Although 20A exhibited rootworm resistance in an insect bioassay, it has never been employed in crosses to maize. Therefore, it is assumed the other loci involved in expression of rootworm resistance must be found in Tripsacorn, Sun Star, 2019, 3024, 3028, 3125, 4126 and TC64. There are three additional candidate loci that have a restriction fragment found only in the rootworm resistant hybrids: BNL5.37 which marks a locus on the long arm of chromosome 3, UMC28 on the long arm of chromosome 6, and UMC95 on the long arm of chromosome 9. This information allows the screening of young seedlings for rootworm resistance without having to go through time-consuming, labor intensive insect bioassays. A small amount of leaf tissue can be used to isolate the genomic DNA from individual plants. The sample can be assayed by RFLP genotyping using the respective enzyme/probe combinations for those four loci or it can be done more rapidly by isolating genomic DNA from small amounts of leaf tissue and genotyping by polymerase chain reaction (PCR) with primers for SSR (simple sequence repeat) markers that have been mapped to corresponding positions as the RFLP markers on the maize chromosomes (see FIG. 2 and Table 5). Plants with two of these marker loci polymorphisms exhibit a degree of resistance to corn rootworm that is equal to or better than the industry standard root rating of 3 for efficacy of insecticide control. Plants with three or more of these RFLP markers have root ratings of 1 or 2 on the Hills and Peters (also referred to as Iowa) scale and are highly resistant (Eubanks 2002).

Simple sequence repeat polymorphisms (SSRs) are rapidly becoming the molecular markers of choice for genotyping, as well as for identifying and mapping genes (Senior et al. 1998), and assessing genetic diversity (Liu et al. 2000). SSRs are intergenic tandem repeats of 2 to 6 base pairs that are amplified by polymerase chain reaction (PCR) using primers complimentary to the flanking regions of the repeats. The PCR products are separated by electrophoresis, and the codominant polymorphisms are visualized as different bands on the gel. SSR variability can be scored as accurately and reliably as RFLP polymorphisms (Smith et al. 1997). Advantages for employing SSRs instead of RFLP markers for marker assisted breeding are they are less labor intensive, less time-consuming, more cost effective, permit rapid, high through-put screening, and require much smaller quantities of DNA. To assess feasibility of using SSRs for marker assisted selection of rootworm resistance a pilot study using 35 SSR markers was conducted to see if they would also amplify the DNA of Tripsacum and Tripsacum-teosinte hybrids. In addition to producing distinct polymorphisms that were inherited from both the Tripsacum and the Zea parents, novel SSR bands were also observed in the Tripsacum-teosinte recombinants and crosses between Tripsacum-teosinte hybrids and maize. SSR markers that map to the same genetic loci as the RFLP markers employed to fingerprint the Tripsacum-teosinte hybrids are listed in Table 5 and indicated beneath each corresponding RFLP marker in FIG. 2. The corresponding SSR markers for the RFLP markers for rootworm resistance are bnlg2235 for UMC103 on the short arm of linkage group 8, dupSSR23 for BNL5.37 on the long arm of linkage group 3, phi123 for UMC28 on the long arm of linkage group 6, and bnlg1714 for UMC95 on the long arm of linkage group 9. The application of SSR marker assisted breeding will greatly facilitate commercial development of maize with natural rootworm resistance imparted from resistant Tripsacum-teosinte recombinants.

In addition to rootworm resistance, another application for using these novel RFLP fragments to select special traits is in regard to transferring constitutive aerenchyma to the roots of maize. Aerenchyma refers to large intercellular spaces in plant tissue that permit internal gas transport between the leaves and roots, and serve as a reservoir of oxygen required for respiration under anaerobic conditions (Esau 1977). Aerenchyma is a common feature of wetland and aquatic plants (Justin and Armstrong 1987), and it occurs in some species adapted to drier environments. Another important function of aerenchyma is diffusion of oxygen into the rhizosphere for oxidation of soil components toxic to plant growth (Armstrong 1979; Drew and Stolzy 1996). Some plants have constitutive aerenchyma that forms early in development. Other plants may gradually develop aerenchyma in response to flooded soil conditions (Justin and Armstrong 1987). The roots of Tripsacum dactyloides possess constitutive aerenchyma (Ray et al. 1998). The air-filled passages in the roots enable gamagrass to grow in saturated soils and to penetrate compacted layers so it can tolerate both floods and droughts (Clark et al. 1996; Foy 1996; Ray et al 1998). The roots can grow deep into subsoils to tap water reserves. Since subsoils are highly acidic, aerenchyma appears to be associated with gamagrass' strong aluminum tolerance (Clark et al. 1996; Foy 1997). Examination of the roots of the Tripsacum dactyloides and Zea diploperennis parents, and Sun Dance Genetics F₁ hybrids under a low power light microscope reveal well developed aerenchyma in all Tripsacum parents but none in any of the teosintes. Aerenchyma is present in Tripsacorn roots but not in those of Sun Dance, Sun Star or 20A. The transfer of constitutive aerenchyma into corn will enhance broad environmental stress tolerance in the world's most widely grown crop. Benefits of commercial development of this technology for American producers, as well as growers worldwide, will be reduced vulnerability to weather extremes of drought as well as the opposite problem of excessive rainfall and standing water. Broad environmental benefits will be reduction of aquifer depletion from irrigation and reduced pollution of waterways and groundwater from irrigation runoff.

Upon examination of a series of roots, aerenchyma was observed in other hybrids including Devil Corn, Sun Devil, 7022, 5 plants of 7022 X Devil Corn, B016, 6021, 4 plants of Zea diploperennis X Tripsacum laxum. Two out of three F₁ plants of Tripsacorn X W64a had aerenchyma. One of two B73 X Tripsacorn plants had intermediate expression of aerenchyma and the other plant had none. This indicates aerenchyma has simple co-dominant inheritance. In a population of 24 SDG058 plants in a breeding program selecting for strong drought tolerance, all had aerenchyma. SDG058 is derived from a B73 X Tripsacorn (ref. 2019 in Tables 2 and 3). Fifteen had well developed aerenchyma indicating they are homozygous for the trait. Roots of plants from three other (Tripsacum-teosinte X maize) hybrid lines that were not selected for drought tolerance (9094 X 7009, 00-2-17, and 99-16-3 did not develop arenchyma. This confirms that the presence of root aerenchyma is contributing to drought tolerance in line SDG058.

SDG058 exhibited strongest drought tolerance when compared to the publicly available corn inbred W64A, the corn parent of SDG058, in controlled environment water deficit experiments. In each of three experiments, 24 plants of each line (12 treatment and 12 control) were planted in 5 gallon pots and placed in the growth chambers in a randomized block design. The plants were watered twice daily until initiation of the water deficit regimen at 42 days after planting, the most critical period in the reproductive and flowering cycle affecting grain yield. The drought period was monitored gravimetrically by weighing the pots daily until they reached a minimum 30% reduction in plant available water. Calculated in pilot tests, this equilibrates to 20% reduction in pot weight. The drought treatment period in these experiments was 5 days with no water and averaged to approximately 30% reduction in pot weight or around 45% reduction in plant available water, a strong drought stress for corn. Grain dry weight was the measure for assaying the degree of drought tolerance. The average SDG058 grain dry weight of plants under drought stress was 198 g per plant. In contrast, the drought stressed W64A corn plants had a yield of 125.2 g per plant. Under drought stress the SDG058 hybrid line outperformed W64A by about 37% greater yield. W64A does not have aerenchyma in its roots. All of the SDG058 plants have root aerenchyma.

Since aerenchyma is present only in Tripsacorn and not in the other Tripsacum-teosinte crosses, unique polymorphisms in Tables 2 and 3 found only in Tripsacorn will signal potential markers for this trait. Aerenchyma is present in the maize X Tripsacum-teosinte plants designated number 2019, 3028 and TC64 in Tables 2 and 3. Logically, the aerenchyma trait will be associated with any marker for unique polymorphisms found only in Tripsacorn, 2019, 3028 and TC64. The only possible marker locus candidate that has an RFLP fragment found exclusively in the hybrids with constitutive aerenchyma is BNL8.32 on the long arm of linkage group 7. Therefore it is concluded that the gene for aerenchyma was transferred to the long arm of Zea chromosome 7. The corresponding SSR marker for the BNL8.32 locus is bnlg2235.

The present invention provides a method of screening plants to determine if they are crosses between Tripsacum and teosinte by isolating their total genomic DNA, digesting the DNA with restriction enzymes, transferring it to Southern blots and probing it with mapped molecular markers to determine the presence of one or more novel or unique RFLPs as defined by probe-enzyme combination and molecular weight. The term “plant” as used in this application refers to the whole plant as well as its component parts, e.g., flowers, roots, fruits, stems, rhizomes, pollen. The crosses are performed using standard plant breeding techniques for controlled pollinations known in the art. Some of the Tripsacum-teosinte hybrid plants that are perennials and reproduce asexually as well as by seed have been described in the following plant patents: PP No. 9,640 issued Sep. 3, 1996; PP No. 7,977 issued Sep. 15, 1992, and PP No. 6,906 issued Jul. 4, 1989. U.S. Pat. No. 5,330,547 issued Jul. 19, 1994, and U.S. Pat. No. 5,750,828 issued May 12, 1998, describe a method for employing Tripsacum-teosinte hybrids to confer corn rootworm resistance to maize.

The present invention further provides a method of screening hybrid maize seed and plants to determine if they contain introgressed DNA segments from Tripsacum-teosinte hybrids by isolating the total genomic DNA, digesting the DNA with restriction enzymes, transferring it to Southern blots and probing it with mapped molecular markers to determine the presence of one or more novel or unique RFLPs as defined by probe-enzyme combination and molecular weight.

The present invention provides a method for marker assisted selection of plants resistant to corn rootworm by the presence of unique DNA fragments revealed by two or more of the RFLP markers identified as UMC103, BNL5.37, UMC28, and UMC 95 and/or their SSR markers bnlg2235, dupSSR23, phi123 and bnlg1714, respectively.

The present invention provides a method for marker assisted selection of plants with aerenchyma tissue in their roots by the presence of a unique DNA fragment revealed by the RFLP marker BNL8.32 and/or its respective SSR marker bnlg1805.

In Tripsacum inflorescences, the staminate (i.e. male) flowers and pistillate (i.e. female) flowers are produced on a single spike with the male flowers subtended by the female. When Tripsacum sends out the inflorescence, the staminate flowers are broken off leaving only the female flowers on the spike which are then covered with a pollinating bag, i.e. standard ear shoot bag for maize, to protect them from contamination by unwanted pollen. Teosinte male and female flowers occur on separate parts of the plant. The staminate flowers are borne in the tassel which emerges at the apex of the culm; whereas, the pistillate flowers occur in single-rowed spikes borne on lateral branches of the culm. When teosinte produces its tassels, they are covered with a pollinating bag. When they start shedding pollen, the bag is removed and pollen taken to pollinate the Tripsacum plants. At that time, the bags covering the Tripsacum pistillate flowers are removed and the teosinte pollen shaken out of the bag onto the silks. The Tripsacum inflorescence is covered again with a pollinating bag immediately after pollination and the bag is stapled so that it remains on the spike until the seed has matured. Upon maturity, approximately 45 days later, the seed is harvested. Once mature seed from the cross has been obtained, it is planted, and the plants from seed that germinates are grown in a growth chamber, greenhouse or the field. Controlled crosses are best made in a greenhouse or growth chamber where plants are kept isolated to prevent cross contamination and there is no problem with bags being damaged by weather conditions.

This method may alternatively be used to cross the plants with teosinte as the female parent. In this embodiment, all the tassels, i.e. male flowers, are removed from the perennial teosinte plant as soon as they emerge and the ears, i.e. female flowers, are covered with pollinating bags. Rather than removing Tripsacum male flowers, the spikes are left in tact and covered with a pollinating bag to collect Tripsacum pollen. The pollen is applied to the diploperennis ears which are then immediately covered with a pollinating bag that is well fastened with staples to ensure it remains sealed until the seed has matured, approximately 45 days after pollination when the seed is harvested.

Next, when (Tripsacum X teosinte) or (teosinte X Tripsacum) starts to flower, the same steps described above are used to cross the hybrid with maize. To cross onto maize, as soon as the maize plants begin to produce ears, before the silks emerge, the ears are covered with an ear shoot bag. Pollen collected from (Tripsacum X teosinte) or (teosinte X Tripsacum) is applied to silks of the maize ears. The ears are then covered again with an ear shoot bag and a large pollinating bag which is wrapped around the culm and secured with a staple. The ears remain covered until they reach maturity, several weeks later when the ears are harvested.

To pollinate the (Tripsacum X teosinte) or (teosinte X Tripsacum) hybrid with maize pollen, the tassel of the maize plant is covered with a large pollinating bag, a day or two before collection. Pistillate flowers of Tripsacum-teosinte hybrid plants frequently have staminate tips above the female flowers as described for Tripsacum. Whenever Tripsacum-teosinte plants are to be pollinated by another plant, all the staminate tips are removed as soon as the ears emerge to prevent possibility of self pollination. The pistillate flowers of the hybrid are covered with an ear shoot bag as soon as they begin to appear on the plant but before the silks emerge. Pollen collected from maize is applied to silks of the hybrid female spikes which are then immediately covered with an ear shoot bag that is stapled closed. The ears remain covered until they reach maturity, approximately 45 days later, and then the seed is harvested.

Plants obtained from all crosses described above are male and female fertile, are cross-fertile with each other, are cross-fertile with maize, and carry novel genetic material, i.e. unique polymorphisms from Tripsacum (see Table 3) that are not present in maize and the wild Zeas and novel restriction fragments (see Table 2) derived from mutations that arose in the process of intergeneric hybridization, as identified in DNA fingerprints employing 176 different molecular probes distributed throughout the ten linkage groups of maize. Table 4 gives the molecular weights of parental RFLPs for comparative reference.

The examples and embodiments described herein are for illustration and modifications or changes that will be suggested to persons skilled in the art are to be included within the spirit and purview of this application and the scope of the appended claims.

TABLE 1
List of RFLP Probes Used to Fingerprint Hybrid and Parent
Plants of Tripsacum, teosinte, Tripsacum-teosinte Hybrids, Maize,
and Maize-Tripsacum-teosinte Hybrids and Derivatives.

| | Chromosome | | | | |
|---|---|---|---|---|---|
| | 1 | 2 | 3 | 4 | 5 |
| Probe | BNL5.62 | UMC53 | UMC32 | aqrr115 | npi409 |
| | npi97 | UMC6 | asg24 | phi20725 | UMC147 |
| | UMC157 | UMC61 | UMC121 | UMC87 | asg73 |
| | UMC76 | aqrr167 | BNL8.35 | UMC31 | UMC90 |
| | UMC11 | UMC34 | UMC50 | UMC55 | UMC72 |
| | asg45 | UMC135 | UMC42 | CSU235 | UMC27 |
| | CSU3 | UMC131 | npi247 | CSU585 | tda66 |
| | UMC167 | | UMC97 | BNL5.46 | UMC43 |
| | UMC67 | UMC55 | UNC10 | aqrr321 | tda37 |
| | CSU92 | | UMC102 | agrr89 | UMC40 |
| | asg62 | UMC5 | BNL6.06 | npi386 | BNL7.71 |
| | UMC58 | | CSU240 | UMC42 | BNL5.71 |
| | CSU164 | | BNL5.37 | tda62 | tda62 |
| | UMC128 | tda66 | npi296 | BNL5.71 | UMC54 |
| | UMC129 | | UMC60 | UMC156 | UMC108 |
| | UMC107 | UMC4 | UMC3 | UMC66 | UMC68 |
| | UMC140 | UMC49 | npi212 | UMC19 | UMC104 |
| | adh1 | UMC36 | UMC39 | UMC104 | phi10017 |
| | UMC161 | | phil10080 | UMC133 | |
| | BNL8.29 | | UMC15 | UMC15 | |
| | BNL6.32 | | UMC63 | UMC52 | |
| | | | CSU303 | BNL8.23 | |
| | | | UMC96 | BNL15.07 | |
| | | | UMC2 | | |
| | | | CSU25 | | |
| | Chromosome | | | | |
| | 6 | 7 | 8 | 9 | 10 |
| Probe | UMC85 | asg8 | npi114 | phi10005 | phi20075 |
| | tda50 | phi20581 | BNL9.11 | UMC113 | BNL3.04 |
| | npi373 | O2 | UMC103 | UMC192 | npi285 |
| | tda204 | asg34 | UMC124 | UMC105 | KSU5 |
| | UMC59 | BNL15.40 | tda52 | CSU147 | UMC130 |
| | npi393 | UMC5 | tda164 | BNL5.10 | UMC64 |
| | UMC65 | UMC116 | UMC32 | UMC114 | UMC152 |
| | tda51 | tda37 | UMC120 | UMC95 | phi06005 |
| | UMC21 | UMC110 | UMC89 | asg44 | tda205 |
| | UMC46 | tda66 | BNL12.30 | CSU61 | |
| | UMC132 | BNL8.32 | UMC30 | BNL7.57 | UMC163 |
| | asg7 | BNL14.07 | UMC48 | BNL5.09 | UMC44 |
| | UMC28 | UMC80 | UMC53 | CSU54 | BNL10.13 |
| | UMC62 | BNL16.06 | npi268 | npi97 | npi306 |
| | UMC134 | phi20020 | npi414 | UMC94 | |
| | | | UMC7 | | |
| | | | UMC3 | | |

Other Probes

| Mitochondrial | | Locus unknown |
|---|---|---|
| Probe | pmt1 | tda16 |
| | pmt2 | tda17 |
| | pmt3 | tda48 |
| | pmt4 | tda53 |
| | pmt5 | tda80 |
| | pmt6 | tda168 |
| | | tda250 |

TABLE 2
Novel RFLPS in Tripsacum-teosinte Hybrids and Maize X
(Tripsacum-teosinte)

| | Tripsacum-teosinte Hybrids | | | | Maize X Tripsacum-teosinte | | | | | | | | | | | | | |
|---|---|---|---|---|---|---|---|---|---|---|---|---|---|---|---|---|---|---|
| Probe/Enzyme | Sun Dance | 20A | Tripsacom | Sun Star | 64SS | 64TC | 2019 | 3024 | 3028 | 3125 | 4126 | TC64 | Sun Devil | 7022 | 7024 | 9094 | 97-5 | V70 |
| Chrom. 1 | | | | | | | | | | | | | | | | | | |
| BNL5.62-ERI | 10.3 kb | | | | | | 10.3 kb | 10.3 kb | 10.3 kb | | | | | | | | | |
| npi97-H | 3.9 kb | | 3.9 kb | | | | 3.9 kb | 3.9 kb | 3.9 kb | 3.9 kb | | | | 3.9 kb | 3.9 kb | | | |
| UMC157-ERI | 6.5 kb | | 6.5 kb | | | 6.5 kb | | | | | | | | | | | | |
| UMC157-ERI | 3.3 kb | | 3.3 kb | | | 3.3 kb | | | | | | | | | | | | |
| UMC157-H | 5.5 kb | | 5.5 kb | | | 5.5 kb | | | | | | | | | | | | |
| UMC157-B | 14.0 kb | 14.0 kb | 14.0 kb | 14.0 kb | 14.0 kb | 14.0 kb | | | 14.0 kb | | | | | | | | | |
| UMC157-B | | 5.0 kb | | 5.0 kb | | | | | 5.0 kb | | | | | 5.0 kb | | | | |
| UMC157-B | | | 4.5 kb | 4.5 kb | | | 4.5 kb | | | | | 4.5 kb | | | 4.5 kb | | | |
| UMC11-B | 7.0 kb | 7.0 kb | 7.0 kb | 7.0 kb | | | 7.0 kb | 7.0 kb | | 7.0 kb | | 7.0 kb | | | | | | |
| CSU3-B | | | 10.0 kb | 10.0 kb | | | 10.0 kb | 10.0 kb | | | 10.0 kb | 10.0 kb | | | | | | |
| CSU3-B | | | 7.6 kb | 7.6 kb | | | 7.6 kb | 7.6 kb | | | 7.6 kb | 7.6 kb | | | | | | |
| CSU3-B | 3.5 kb | | 3.5 kb | | | | | | | 3.5 kb | | 3.5 kb | | | | | | |
| UMC67-ERI | | | | 19.2 kb | | | | | | | | | | | | | | |
| UMC67-H | | | | 23.1 kb | | | | | | | | | | | | | | |
| UMC67-B | 13.4 kb | | | | | | | | | | | | | | | | | |
| UMC67-B | 11.0 kb | 11.0 kb | 11.0 kb | 11.0 kb | | | 11.0kb | 11.0kb | 11.0 kb | 11.0 kb | 11.0 kb | 11.0 kb | | | | | | |
| UMC67-B | | | | 1.6 kb | | | | | | | | | | | | | | |
| CSU92-B | 13.3 kb | | 13.3 kb | 13.3 kb | | | 13.3 kb | 13.3 kb | | | | 13.3 kb | | | | | | |
| CSU92-B | 7.5 kb | 7.5 kb | | | | | | | | | | | | | | | | |
| asg62-B | 12.7 kb | 12.7 kb | 12.7 kb | | | | | | | | | | | | | | | |
| asg62-B | | 9.7 kb | 9.7 kb | 9.7 kb | | | 9.7 kb | 9.7 kb | 9.7 kb | | 9.7 kb | | | | | | | |
| asg62-B | 6.6 kb | | 6.6 kb | | | | | | | | | | | | | | | |
| UMC58-H | 3.3 kb | | 3.3 kb | 3.3 kb | 3.3 kb | 3.3 kb | | | | | | | | | | | | |
| CSU164-ERI | 9.0 kb | | 9.0 kb | 9.0 kb | | | 9.0 kb | 9.0 kb | | | 9.0 kb | | | | | | | |
| CSU164-ERI | | 7.0 kb | | | | | | | | | | | | | | | | |
| UMC128-H | | | 6.0 kb | 6.0 kb | | | 6.0 kb | 6.0 kb | | | 6.0 kb | 6.0 kb | | | | | | |
| UMC107-ERI | | | 6.3 kb | 6.3 kb | 6.3 kb | | | | | | | | | | | | | |
| UMC107-ERI | | | 6.1 kb | | | | 6.1 kb | 6.1 kb | | 6.1 kb | | | | | | | | |
| UMC140-ERI | 4.9 kb | | 4.9 kb | 4.9 kb | | | 4.9 kb | 4.9 kb | 4.9 kb | | | 4.9 kb | | | | 4.9 kb | | |
| UMC140-H | 6.5 kb | | 6.5 kb | | | | | | | | | | | | | | | |
| adh1-H | 9.4 kb | | 9.4 kb | 9.4 kb | | | | | | | | | | | | | | |
| adh1-B | 9.4 kb | | 9.4 kb | 9.4 kb | 9.4 kb | 9.4 kb | | | | | | | | | | | | |
| UMC161-H | 3.3 kb | | 3.3 kb | 3.3 kb | 3.3 kb | 3.3 kb | | | | | | | | | | | | |
| BNL8.29-H | | | 9.3 kb | | | | | | | | 9.3 kb | | | | | | | |
| BNL8.29-H | 8.3 kb | 8.3 kb | 8.3 kb | 8.3 kb | | | | | | | | 8.3 kb | | | | | | |
| Chrom. 2 | | | | | | | | | | | | | | | | | | |
| UMC53-ERI | 9.4 kb | | 9.4 kb | 9.4 kb | 9.4 kb | | | | | | | | | | | | | |
| UMC53-ERV | | 3.8 kb | 3.8 kb | 3.8 kb | | | 3.8 kb | 3.8 kb | 3.8 kb | | | 3.8 kb | | | | | | |
| UMC53-ERV | 3.0 kb | | | | | | | | | | | | | | | | | |
| UMC6-ERI | | | 3.8 kb | | | | | | | | | | | | | | | |
| UMC6-H | | | 9.4 kb | 9.4 kb | 9.4 kb | | | | | | | | | | | | | |
| UMC6-B | | | 15.3 kb | | | | | | | | | | | | | | | |
| UMC6-B | 13.2 kb | 13.2 kb | | | | | | | | | | | | | | | | |
| UMC6-B | | | 12.7 kb | | | | | | | | | | | | | | | |
| UMC6-B | | 10.0 kb | | | | | | | | | | | | | | | | |
| UMC6-B | 7.0 kb | 7.0 kb | | | | | 7.0 kb | 7.0 kb | 7.0 kb | 7.0 kb | 7.0 kb | | 7.0 kb | | | | | |
| UMC61-H | | | 3.4 kb | 3.4 kb | | | | | | | | | | | | | | |
| UMC61-H | 2.8 kb | | 2.8 kb | 2.8 kb | | | 2.8 kb | 2.8 kb | 2.8 kb | 2.8 kb | 2.8 kb | 2.8 kb | | | | | | |
| UMC34-ERI | 7.5 kb | | | | | | | | | | | | | | | | | |
| UMC34-ERI | 5.4 kb | | 5.4 kb | 5.4 kb | 5.4 kb | | | | | | | | | | | | | |
| UMC34-H | 8.8 kb | | 8.8 kb | 8.8 kb | 8.8 kb | | | | | | | | | | | | | |
| UMC34-H | 6.5 kb | | 6.5kb | | | | | | | | | | | | | | | |
| UMC34-H | 5.8 kb | 5.8 kb | | | | | 5.8 kb | 5.8 kb | | 5.8 kb | 5.8 kb | 5.8 kb | | | | | | |
| UMC34-B | | | 9.4 kb | 9.4 kb | 9.4 kb | | | | | | | | | | | | | |
| UMC135-H | | 11.6 kb | | | | | | | | | | | | | | | | |
| UMC135-H | | | 10.8 kb | | | | 10.8 kb | 10.8 kb | | 10.8 kb | | | | | | | | |
| UMC131-ERI | | | 10.6 kb | | | | | | | | | | | | | | | |
| UMC131-ERI | 5.8 kb | | | | | | | | | 5.8 kb | 5.8 kb | | | | | | | |
| UMC131-ERI | 4.3 kb | 4.3 kb | 4.3 kb | 4.3 kb | 4.3 kb | 4.3 kb | 4.3 kb | 4.3 kb | 4.3 kb | | | | | | | | | |
| UMC55-ERI | 3.9 kb | 3.9 kb | 3.9 kb | | | | 3.9 kb | 3.9 kb | | 3.9 kb | 3.9 kb | 3.9 kb | | | | | | |
| UMC55-H | 4.3 kb | | 4.3 kb | | | | | | | | | | | | | | | |
| UMC5-ERI | 5.4 kb | | 5.4 kb | 5.4 kb | 5.4 kb | | | | | | | | | | | | | |
| UMC5-H | | | 6.5 kb | 6.5 kb | 6.5 kb | | | | | | | | | | | | | |
| UMC49-B | | 8.2 kb | 8.2 kb | 8.2 kb | | | | | | | | | | | | | | |
| UMC36-B | 4.2 kb | | | | | | | | | | | | | | | | | |
| Chrom. 3 | | | | | | | | | | | | | | | | | | |
| UMC32-ERI | 5.3 kb | | 5.3 kb | 5.3 kb | 5.3 kb | 5.3 kb | 6.7 kb | 6.7 kb | | 6.7 kb | 6.7 kb | 6.7 kb | | | | | | |
| UMC32-H | 6.7 kb | | 6.7 kb | 6.7 kb | | | | | | | | | | | | | | |
| UMC32-H | | | 6.0 kb | | | | | | | | | | | | | 6.0 kb | | |
| UMC32-H | | | 2.8 kb | | | | | | | | | | | | | | | |
| asg24-H | 7.2 kb | 7.2 kb | 7.2 kb | 7.2 kb | | | | | 7.2 kb | 7.2 kb | 7.2 kb | 7.2 kb | | | | | | |
| asg24-H | 6.4 kb | | 6.4 kb | 6.4 kb | | | | | | 6.4 kb | | 6.4 kb | | | | | | |
| UMC121-ERI | 3.7 kb | 3.7 kb | 3.7 kb | 3.7 kb | | | | 3.7 kb | 3.7 kb | | | 3.7 kb | | | | 3.7 kb | 3.7 kb | |
| UMC121-ERI | 3.2 kb | 3.2 kb | 3.2 kb | 3.2 kb | | | | | | | | | | 3.2 kb | | | | |
| BNL8.35-H | | | 9.9 kb | | | | | | | | | | | | | | | |
| BNL8.35-H | | 8.7 kb | | | | | | | | | | | | | | | | |
| UMC50-B | 6.8 kb | | | | | | | | | | | | | | | | | |
| UMC50-B | | | 3.8 kb | | | | | | 3.8 kb | 3.8 kb | 3.8 kb | | 3.8 kb | | | | | |
| UMC42-H | 10.4 kb | 10.4 kb | 10.4 kb | 10.4 kb | | | 10.4 kb | 10.4 kb | 10.4 kb | 10.4 kb | 10.4 kb | 10.4 kb | | | | | | |
| UMC42-H | | | 9.2 kb | 9.2 kb | | | | | | | | | | 9.2 kb | 9.2 kb | | | |
| UMC42-H | | 8.9 kb | | 8.9 kb | | | | | 8.9 kb | | | | | | | | | |
| UMC42-H | | | 7.9 kb | | | | | | | | | | | 7.9 kb | | 7.9 kb | 7.9 kb | |
| UMC42-H | 3.7 kb | | 3.7 kb | | | | 3.7 kb | 3.7 kb | 3.7 kb | 3.7 kb | 3.7 kb | 3.7 kb | | | | | | |
| UMC42-H | 3.0 kb | | | | | | | | | | | | | | | | | |
| npi247-ERI | 8.0 kb | | 8.0 kb | 8.0 kb | 8.0 kb | | | | | | | | | | | | | |
| npi247-H | | | 3.0 kb | 3.0 kb | 3.0 kb | | | | | | | | | | | | | |
| UMC10-ERI | | | 6.5 kb | 6.5 kb | 6.5 kb | | | | | | | | | | | | | |
| UMC10-ERI | | | 5.5 kb | 5.5 kb | 5.5 kb | | | | | | | | | | | | | |
| UMC10-H | 3.0 kb | | | 3.0 kb | | | | | | | | | | | | | | |
| UMC102-ERI | | 2.7 kb | | 2.7 kb | | | | | 2.7 kb | | | | | | | | | |
| BNL6.06-ERI | 6.8 kb | 6.8 kb | 6.8 kb | 6.8 kb | | | | 6.8 kb | 6.8 kb | | | 6.8 kb | | | | | | |
| CSU240-ERI | | | 10.6 kb | 10.6 kb | | | | | | | | | | | | | | |
| CSU240-ERI | | | 4.5 kb | 4.5 kb | | | | | | | | | | | | | | |
| CSU240-ERI | | | 3.3 kb | 3.3 kb | | | | | | | | | | 3.3 kb | | 3.3 kb | | 3.3 kb |
| BNL5.37-H | | | 10.3 kb | 10.3 kb | | | 10.3 kb | 10.3 kb | 10.3 kb | 10.3 kb | 10.3 kb | 10.3 kb | | | | | | |
| BNL5.37-H | 5.8 kb | 5.8 kb | 5.8 kb | 5.8 kb | | | | | 5.8 kb | | | 5.8 kb | | | | | | |
| BNL5.37-H | 3.5 kb | 3.5 kb | 3.5 kb | 3.5 kb | | | 3.5 kb | 3.5 kb | 3.5 kb | 3.5 kb | 3.5 kb | 3.5 kb | | | | | | |
| npi296-ERI | 7.9 kb | | 7.9 kb | 7.9 kb | 7.9 kb | 7.9 kb | | | | | | | | | | | | |
| UMC3-ERI | 2.5 kb | | 2.5 kb | 2.5 kb | | | 2.5 kb | 2.5 kb | | 2.5 kb | 2.5 kb | 2.5 kb | | | | | | |
| UMC3-ERI | 2.0 kb | 2.0 kb | | 2.0 kb | | | | | | | | | | | | | | |
| npi212-H | | | 4.3 kb | 4.3 kb | | | | | | | | | | | | | | |
| npi212-B | | | 5.4 kb | 5.4 kb | | | | | | | | | | | | | | |
| UMC39-ERI | 12.2 kb | 12.2 kb | | | | | | | 12.2 kb | | | | | | | | | |
| UMC39-ERI | | 9.2 kb | | 9.2 kb | | | | | 9.2 kb | | | | | | | | | |
| UMC39-ERI | 7.8 kb | 7.8 kb | | 7.8 kb | | | | | | | | | | | | | | |
| UMC39-ERI | 7.1 kb | | 7.1 kb | 7.1 kb | | | | 7.1 kb | | 7.1 kb | 7.1 kb | 7.1 kb | | | | | | |
| CSU303-ERI | | | 10.0 kb | 10.0 kb | | | | | | | | | | | | 10.0 kb | | |
| UMC63-H | | | | 9.5 kb | | | 9.5 kb | | 9.5 kb | 9.5 kb | 9.5 kb | 9.5 kb | | | | | | |
| UMC63-H | 4.3 kb | 4.3 kb | | 4.3 kb | | | | | 4.3 kb | | | | | | | | | |
| UMC96-H | | | 11.8 kb | 11.8 kb | | | 11.8 kb | 11.8 kb | | | | | | | | | | |
| UMC96-H | | | | 6.4 kb | | | | | 6.4 kb | | | | | | | | | |
| UMC96-H | 5.5 kb | | 5.5 kb | 5.5 kb | | | | | | | | | | | | | | |
| UMC96-B | 7.5 kb | | | | | | | | | | | | | | | | | |
| UMC2-ERI | 11.8 kb | | 11.8 kb | | | | | | | 11.8 kb | 11.8 kb | | | | | | | |
| UMC2-ERI | | | | 10.4 kb | | | | | | | | | | | | | | |
| UMC2-ERI | | 8.0 kb | | 8.0 kb | | | | | 8.0 kb | | | | | | | | | |
| UMC2-ERI | | | | 3.9 kb | | | | | | | | | | | | | | |
| CSU25-H | 4.5 kb | | | | | | | | | 4.5 kb | 4.5 kb | 4.5 kb | | | | | | |
| Chrom. 4 | | | | | | | | | | | | | | | | | | |
| agrr115-ERI | | | 8.0 kb | 8.0 kb | 8.0 kb | 8.0 kb | | | | | | | | | | | | |
| agrr115-ERI | 5.4 kb | | 5.4 kb | | 5.4 kb | 5.4 kb | | | | | | | | | | | | |
| agrr115-H | | | 19.2 kb | 19.2 kb | 19.2 kb | | | | | | | | | | | | | |
| agrr115-B | | | 5.4 kb | 5.4 kb | 5.4 kb | 5.4 kb | | | | | | | | | | | | |
| agrr115-B | | | 3.5 kb | 3.5 kb | 3.5 kb | 3.5 kb | | | | | | | | | | | | |
| phi20725-ERI | | | 10.3 kb | 10.3 kb | | | | | | | | 10.3 kb | 10.3 kb | | | | | |
| phi20725-ERI | | 7.2 kb | | 7.2 kb | | | | 7.2 kb | | | | | | | | | | |
| phi20725-H | 1.5 kb | | | | | | | | | 1.5 kb | 1.5 kb | | | | | | | |
| UMC31-ERI | | | 5.8 kb | 5.8 kb | 5.8 kb | | | | | | | | | | | | | |
| UMC31-ERI | | | 2.0 kb | 2.0 kb | | | | | | | | | 2.0 kb | | 2.0 kb | | | |
| UMC55-ERI | 3.9 kb | 3.9 kb | 3.9 kb | | | | 3.9 kb | 3.9 kb | | 3.9 kb | 3.9 kb | 3.9 kb | | | | | | |
| UMC55-H | 4.3 kb | | 4.3 kb | | | | | | | | | | | | | | | |
| CSU235-H | | | 6.8 kb | 6.8 kb | | | | | | | | | | 6.8 kb | | 6.8 kb | | |
| CSU235-H | | | 3.0 kb | | | | | | | | | | | | | 3.0 kb | | |
| CSU585-H | | | 8.3 kb | 8.3 kb | | | | | | | | | | | | 8.3 kb | | |
| CSU585-H | | | 6.1 kb | 6.1 kb | | | | | | | | | | | | | | |
| BNL5.46-H | 13.7 kb | | | | | | | | | | | | | | | | | |
| BNL5.46-H | | 10.5 kb | | 10.5 kb | | | | | | | | | | | | | | |
| BNL5.46-H | 9.7 kb | | 9.7 kb | 9.7 kb | | | 9.7 kb | 9.7 kb | 9.7 kb | 9.7 kb | 9.7 kb | 9.7 kb | | | | | | |
| BNL5.46-H | | | 5.1 kb | | | | | | 5.1 kb | | | | | | | | | |
| agrr321-B | | | 5.5 kb | 5.5 kb | | | | | | | | | | 5.5 kb | 5.5 kb | 5.5 kb | 5.5 kb | 5.5 kb |
| npi386-H | 9.3 kb | 9.3 kb | | | | | | | | | | | | | | | | |
| npi386-H | 8.2 kb | | 8.2 kb | | | | | | | 8.2 kb | | | | | | | | |
| UMC42-H | 19.2 kb | | 19.2 kb | 19.2 kb | 19.2 kb | 19.2 kb | | | | | | | | | | | | |
| UMC42-H | 10.3 kb | 10.3 kb | 10.3 kb | 10.3 kb | | | 10.3 kb | 10.3 kb | 10.3 kb | 10.3 kb | 10.3 kb | 10.3 kb | 10.3 kb | | | | | |
| UMC42-H | | 8.9 kb | | 8.9 kb | | | | | 8.9 kb | | | | | | | | | |
| UMC42-H | 3.7 kb | | 3.7 kb | | | | 3.7 kb | 3.7 kb | 3.7 kb | 3.7 kb | 3.7 kb | 3.7 kb | | | | | | |
| UMC42-H | 3.0 kb | | | | | | | | | | | | | | | | | |
| tda62-B | 5.5 kb | 5.5 kb | 5.5 kb | | | | | | | 5.5 kb | | | | | | | | |
| tda62-B | | | 5.2 kb | | | | | | | | | | | | | 5.2 kb | 5.2 kb | |
| tda62-B | 4.8 kb | | 4.8 kb | 4.8 kb | | | | | | | | | | | | | | |
| BNL5.71-ERV | 11.3 kb | 11.3 kb | 11.3 kb | 11.3 kb | | | | | | | | 11.3 kb | | | | | | |
| BNL5.71-ERV | | 6.8 kb | | 6.8 kb | | | | | 6.8 kb | | | | | | | | | |
| BNL5.71-ERV | 5.7 kb | | 5.7 kb | | | | 5.7 kb | 5.7 kb | | 5.7 kb | 5.7 kb | 5.7 kb | | | | | | |
| UMC156-H | 3.0 kb | | 3.0 kb | 3.0 kb | 3.0 kb | | | | | | | | | | | | | |
| UMC66-ERI | | | 10.5 kb | 10.5 kb | | | | | | | | | | | | | | |
| UMC66-B | 3.7 kb | 3.7 kb | 3.7 kb | 3.7 kb | | | 3.7 kb | 3.7 kb | 3.7 kb | 3.7 kb | 3.7 kb | 3.7 kb | | | | | | |
| UMC66-B | | | 2.4 kb | 2.4 kb | | | | | | | | | | 2.4 kb | 2.4 kb | 2.4 kb | 2.4 kb | |
| UMC19-B | 12.3 kb | 12.3 kb | 12.3 kb | 12.3 kb | | | 12.3 kb | 12.3 kb | 12.3 kb | | 12.3 kb | 12.3 kb | | | | | | |
| UMC104-H | | | | 12.4 kb | | | | | | | | | | | | | | |
| UMC104-H | 11.6 kb | | 11.6 kb | | | | | | | | | | | | | | | |
| UMC104-H | | 7.5 kb | | | | | | | | | | | | | | | | |
| UMC104-B | 9.4 kb | | 9.4 kb | 9.4 kb | 9.4 kb | 9.4 kb | | | | | | | | | | | | |
| UMC133-H | 10.6 kb | 10.6 kb | 10.6 kb | | | | | | | | | | | | | | | |
| UMC133-H | | | | 9.9 kb | | | | | | | | | | | | | | |
| UMC133-H | | | 9.2 kb | | | | | 9.2 kb | 9.2 kb | 9.2 kb | 9.2 kb | | | | | | | |
| UMC133-H | | | | 7.7 kb | | | | | | | | | | | | | | |
| UMC52-B | | | 8.7 kb | 8.7 kb | | | 8.7 kb | 8.7 kb | | 8.7 kb | 8.7 kb | 8.7 kb | | | | | | |
| UMC52-B | 6.9 kb | | | | | | | | | | | | | | | | | |
| UMC52-B | 3.8 kb | | 3.8 kb | | | 3.8 kb | | | | | | | 3.8 kb | | | | | |
| UMC52-B | 3.0 kb | | | | | | | | | | | | 3.0 kb | | | | | |
| UMC52-B | | | 2.0 kb | 2.0 kb | 2.0 kb | | | | | | | | | | | | | |
| BNL15.07-H | 2.9 kb | | | | | | | | | | | | | | | | | |
| BNL15.07-H | | | | 2.7 kb | | | | | | | | | | | | | | |
| Chrom. 5 | | | | | | | | | | | | | | | | | | |
| npi409-ERI | 9.4 kb | | 9.4 kb | | | | | | | | | | | | | | | |
| npi409-H | | | 10.4 kb | | | | | | | | | | | | | | | |
| npi409-H | | | 9.0 kb | 9.0 kb | 9.0 kb | | | | | | | | 9.0 kb | | | | | |
| npi409-H | 3.9 kb | 3.9 kb | 3.9 kb | 3.9 kb | | 3.9 kb | 3.9 kb | 3.9 kb | 3.9 kb | 3.9 kb | 3.9 kb | 3.9 kb | 3.9 kb | | | | | |
| npi409-H | 3.0 kb | | 3.0 kb | 3.0 kb | 3.0 kb | 3.0 kb | | | | | | | 3.0 kb | | | | | |
| npi409-B | | | 19.2 kb | 19.2 kb | 19.2 kb | | | | | | | | | | | | | |
| UMC147-H | | 16.3 kb | | 16.3 kb | | | 16.3 kb | | 16.3 kb | | | | | | | | | |
| UMC147-H | 3.8 kb | | | | | | | | | | | | | | | | | |
| UMC147-H | 2.4 kb | | 2.4 kb | | | | 2.4 kb | 2.4 kb | | 2.4 kb | | | | | | | | |
| UMC90-H | | | 6.5 kb | 6.5 kb | | | | | | | | | | | 6.5 kb | 6.5 kb | | |
| UMC90-H | 2.8 kb | | | | | | | | | | | | | | | | | |
| UMC90-H | 1.6 kb | | | | | | | | | | | | | | | | | |
| UMC90-B | | | 9.0 kb | 9.0 kb | 9.0 kb | | | | | | | | | | | | | |
| UMC107-ERI | 6.3 kb | | | 6.3 kb | | | | | 6.3 kb | | | | | | | | | |
| UMC27-H | 4.5 kb | | | | | | | | | | | | | | | | | |
| UMC27-B | 6.5 kb | | | | | | | | | | | | | | | | | |
| tda37-B | 8.0 kb | | | | | | | | | 8.0 kb | | | | | | | | |
| tda37-B | 6.4 kb | | | | | | | | | 6.4 kb | | | | | | | | |
| UMC43-B | | 9.7 kb | | 9.7 kb | | | | | 9.7 kb | | | | | | | | | |
| UMC43-B | 5.7 kb | | | | | | | | | | | | | | | | | |
| UMC40-B | 7.2 kb | | | | | | | | | | | | | | | | | |
| UMC40-B | | 4.7 kb | 4.7 kb | 4.7 kb | | | 4.7 kb | | | | | | | 4.7 kb | | 4.7 kb | | 4.7 kb |
| UMC40-B | | | 4.3 kb | 4.3 kb | | | 4.3 kb | | | | 4.3 kb | 4.3 kb | | | | | | |
| BNL7.71-H | | | 10.6 kb | | | | | | | | 10.6 kb | 10.6 kb | | | | | | |
| BNL5.71-B | 11.3 kb | 11.3 kb | 11.3 kb | 11.3 kb | | | | | | | | 11.3 kb | | | | | | |
| BNL5.71-B | | 6.8 kb | | 6.8 kb | | | | | 6.8 kb | | | | | | | | | |
| BNL5.71-B | 5.7 kb | | 5.7 kb | | | | 5.7 kb | 5.7 kb | | 5.7 kb | 5.7 kb | 5.7 kb | | | | | | |
| tda62-B | | | 6.5 kb | 6.5 kb | | | | | 6.5 kb | | | | | | | 6.5 kb | | |
| tda62-B | 5.5 kb | 5.5 kb | 5.5 kb | | | | | | | 5.5 kb | | | | | | | | |
| UMC68-H | 6.0 kb | 6.0 kb | 6.0 kb | 6.0 kb | | | 6.0 kb | 6.0 kb | 6.0 kb | | 6.0 kb | 6.0 kb | | | | | | |
| UMC104-H | | | | 12.4 kb | | | | | | | | | | | | | | |
| UMC104-H | 11.6 kb | | 11.6 kb | | | | | | | | | | | | | | | |
| UMC104-H | | 7.5 kb | | | | | | | | | | | | | | | | |
| UMC104-B | 9.4 kb | | 9.4 kb | 9.4 kb | 9.4 kb | 9.4 kb | | | | | | | 9.4 kb | | | | | |
| phi10017-B | | | 15.1 kb | | | | | | | | | | | | | | | |
| phi10017-B | 9.5 kb | 9.5 kb | 9.5 kb | 9.5 kb | | | 9.5 kb | 9.5 kb | 9.5 kb | | 9.5 kb | | | | | | | |
| Chrom. 6 | | | | | | | | | | | | | | | | | | |
| tda50-B | 8.5 kb | 8.5 kb | 8.5 kb | 8.5 kb | | | | 8.5 kb | 8.5 kb | 8.5 kb | 8.5 kb | 8.5 kb | | 8.5 kb | | 8.5 kb | | 8.5 kb |
| npi373-H | 6.5 kb | | 6.5 kb | 6.5 kb | | | 6.5 kb | 6.5 kb | | 6.5 kb | 6.5 kb | 6.5 kb | | | | | | |
| npi373-H | | 5.6 kb | | 5.6 kb | | | 5.6 kb | 5.6 kb | 5.6 kb | | | | | 5.6 kb | | | | |
| npi373-H | | | 5.1 kb | 5.1 kb | | | | | | | | | | | | 5.1 kb | | 5.1 kb |
| npi373-H | 3.0 kb | | | | | | | | | | | | | 3.0 kb | | 3.0 kb | 3.0 kb | 3.0 kb |
| tda204-B | | | 4.0 kb | | | | 4.0 kb | 4.0 kb | | | | 4.0 kb | | | | 4.0 kb | | |
| NPI393-ERI | 12.1 kb | | 12.1 kb | | | | 12.1 kb | 12.1 kb | | 12.1 kb | 12.1 kb | | | | | | | |
| NPI393-ERI | 8.5 kb | 8.5 kb | 8.5 kb | 8.5 kb | | | | | 8.5 kb | | | 8.5 kb | | | | | | |
| NPI393-ERI | 5.6 kb | 5.6 kb | | | | | | | | | | | | | | | | |
| UMC65-H | 2.9 kb | | | | | | | | | | | | | | | | | |
| UMC46-ERI | 6.5 kb | | 6.5 kb | 6.5 kb | 6.5 kb | 6.5 kb | | | | | | | | 6.5 kb | 6.5 kb | | | |
| UMC46-ERI | 5.6 kb | 5.6 kb | 5.6 kb | 5.6 kb | | | 5.6 kb | 5.6 kb | 5.6 kb | 5.6 kb | 5.6 kb | 5.6 kb | | 5.6 kb | 5.6 kb | 5.6 kb | | 5.6 kb |
| asg7-H | 6.3 kb | | | | | | | | | | | | | | | | | |
| UMC28-H | | | 15.8 kb | 15.8 kb | | | | | | | | | | | | | | |
| UMC28-H | | | 11.9 kb | 11.9 kb | | | | | | | | | | | | | | |
| UMC28-B | | | 7.6 kb | 7.6 kb | | | 7.6 kb | 7.6 kb | 7.6 kb | 7.6 kb | 7.6 kb | 7.6 kb | | | | | | |
| UMC28-B | | | 6.6 kb | 6.6 kb | | | 6.6 kb | | | | | | | | | | | |
| UMC134-ERI | 15.3 kb | | 15.3 kb | 15.3 kb | 15.3 kb | 15.3 kb | | | | | | | | | | | | |
| UMC134-H | 7.5 kb | | 7.5 kb | 7.5 kb | 7.5 kb | 7.5 kb | | | | | | | | | | | | |
| UMC134-H | 4.7 kb | 4.7 kb | 4.7 kb | 4.7 kb | | | | | 4.7 kb | | | | | | | 4.7 kb | | |
| Chrom. 7 | | | | | | | | | | | | | | | | | | |
| asg8-H | 10.8 kb | | 10.8 kb | | | | | | | | | | | | | | | |
| asg8-H | | 8.4 kb | 8.4 kb | 8.4 kb | | | | | | | | | | | | | | |
| phi20581-H | | | 4.2 kb | 4.2 kb | | | | | | | | | | | | 4.2 kb | | |
| O2-ERI | 9.4 kb | | 9.4 kb | 9.4 kb | | | | | | | | | 9.4 kb | | | | | |
| asg34-H | | | 4.5 kb | | | | | | | | | | | 4.5 kb | | 4.5 kb | | |
| BNL15.40-H | 5.8 kb | 5.8 kb | 5.8 kb | | | | | | | | 5.8 kb | | | | | | | |
| UMC116-ERI | 9.5 kb | 9.5 kb | 9.5 kb | 9.5 kb | | | | | | | 9.5 kb | | | | | | | |
| UMC116-H | 15.3 kb | | | | | | | | | | | | | | | | | |
| UMC110-B | 10.6 kb | 10.6 kb | 10.6 kb | 10.6 kb | | | 10.6 kb | 10.6 kb | 10.6 kb | 10.6 kb | 10.6 kb | 10.6 kb | | | | | | |
| UMC110-B | 4.9 kb | 4.9 kb | 4.9 kb | 4.9 kb | | | 4.9 kb | 4.9 kb | | 4.9 kb | 4.9 kb | | | | | | | |
| BNL8.32-H | | | 8.9 kb | | | | 8.9 kb | | 8.9 kb | | | 8.9 kb | | | | | | |
| BNL8.32-H | 7.4 kb | | | | | | | | | | | | | | | | | |
| BNL8.32-H | | 7.1 kb | 7.1 kb | 7.1 kb | | | | | | | | | | | | | | |
| BNL14.07-ERI | | 6.4 kb | | 6.4 kb | | | | | | | | | | | | | | |
| UMC80-H | 2.4 kb | 2.4 kb | | 2.4 kb | | | | 2.4 kb | 2.4 kb | | | | | | | | | |
| BNL16.06-ERI | 6.8 kb | 6.8 kb | 6.8 kb | 6.8 kb | | | | 6.8 kb | 6.8 kb | | | 6.8 kb | | | | | | |
| BNL16.06-H | 5.7 kb | 5.7 kb | 5.7 kb | 5.7 kb | | | 5.7 kb | | 5.7 kb | | | | | 5.7 kb | 5.7 kb | 5.7 kb | 5.7 kb | 5.7 kb |
| BNL16.06-H | | | 3.0 kb | 3.0 kb | | | | | | | | | | | | | | |
| BNL16.06-H | 1.6 kb | 1.6 kb | 1.6 kb | 1.6 kb | | | 1.6 kb | 1.6 kb | 1.6 kb | 1.6 kb | 1.6 kb | 1.6 kb | | 1.6 kb | 1.6 kb | 1.6 kb | | |
| phi20020-H | 7.8 kb | 7.8 kb | 7.8 kb | 7.8 kb | | | 7.8 kb | 7.8 kb | 7.8 kb | 7.8 kb | 7.8 kb | 7.8 kb | | 7.8 kb | 7.8 kb | 7.8 kb | | 7.8 kb |
| phi20020-H | 6.6 kb | | | | | | | | | | | | | | | | | |
| phi20020-H | | | 5.1 kb | 5.1 kb | | | | | | | | | | | | | | |
| Chrom. 8 | | | | | | | | | | | | | | | | | | |
| npi114-H | | | 10.0 kb | 10.0 kb | | | | | | 10.0 kb | 10.0 kb | 10.0 kb | | | | | | |
| npi114-H | 8.8 kb | | 8.8 kb | 8.8 kb | | | | 8.8 kb | 8.8 kb | | | 8.8 kb | | | | | | |
| npi114-H | | | 6.3 kb | | | | | | | | | 6.3 kb | | | | | | |
| BNL9.11-H | | 3.4 kb | | | | | | | | | | | | | | | | |
| UMC103-H | | 6.9 kb | 6.9 kb | 6.9 kb | | | 6.9 kb | 6.9 kb | 6.9 kb | 6.9 kb | 6.9 kb | 6.9 kb | 6.9 kb | | | | | |
| UMC124-H | | | 8.0 kb | 8.0 kb | 8.0 kb | 8.0 kb | | | | | | | | | | | | |
| UMC124-H | 7.0 kb | | 7.0 kb | | | 7.0 kb | | | | | | | | | | | | |
| UMC124-B | 21.0 kb | | 21.0 kb | 21.0 kb | 21.0 kb | 21.0 kb | | | | | | | | | | | | |
| UMC124-B | 19.0 kb | | 19.0 kb | 19.0 kb | 19.0 kb | 19.0 kb | | | | | | | | | | | | |
| UMC124-B | | | 6.6 kb | 6.6 kb | | | | | | | | | | | | | | |
| UMC124-B | | 2.6 kb | 2.6 kb | 2.6 kb | | | 2.6 kb | 2.6 kb | | | | 2.6 kb | | | | | | |
| UMC124-B | 1.6 kb | 1.6 kb | 1.6 kb | 1.6 kb | | | 1.6 kb | 1.6 kb | 1.6 kb | 1.6 kb | 1.6 kb | 1.6 kb | | | | | | |
| UMC120-H | | | 8.0 kb | 8.0 kb | 8.0 kb | 8.0 kb | | | | | | | | | | | | |
| UMC120-H | 3.2 kb | | 3.2 kb | 3.2 kb | | | 3.2 kb | | 3.2 kb | 3.2 kb | 3.2 kb | 3.2 kb | | | | | | |
| UMC120-H | 2.3 kb | | | | | | | | | | | | | | | | | |
| UMC120-H | | 1.4 kb | 1.4 kb | 1.4 kb | | | | | | | | 1.4 kb | | | | | | |
| UMC120-B | | | 23.1 kb | | | | | | | | | | | | | | | |
| UMC89-ERI | | | | 7.3 kb | | | | | | | | | | | | | | |
| UMC89-H | | | | 7.3 kb | | | | | | | | | | | | | | |
| UMC89-B | | | 9.5 kb | 9.5 kb | 9.5 kb | | 9.5 kb | 9.5 kb | | | | 9.5 kb | | | | | | |
| UMC89-B | | 6.0 kb | | 6.0 kb | | | | | 6.0 kb | | | | | | | | | |
| UMC89-B | 5.2 kb | | 5.2 kb | | | | | | | 5.2 kb | 5.2 kb | 5.2 kb | | | | | | |
| UMC89-B | 4.5 kb | | 4.5 kb | 4.5 kb | | | | | | 4.5 kb | 4.5 kb | 4.5 kb | | | | | | |
| UMC89-Mspl | | | 6.7 kb | 6.7 kb | | | | | | | | | | 6.7 kb | | | | |
| UMC89-Mspl | | | 5.8 kb | 5.8 kb | | | | | | | | | | 5.8 kb | | 5.8 kb | 5.8 kb | 5.8 kb |
| BNL12.30-ERI | | 3.5 kb | | | | | | | | | | | | | | | | |
| UMC48-H | | | 5.3 kb | 5.3 kb | | | | | | | | | | 5.3 kb | | 5.3 kb | | |
| UMC48-H | | | | 4.7 kb | | | 4.7 kb | | 4.7 kb | | | | | | | | | |
| UMC48-H | | | 4.2 kb | 4.2 kb | | | | | | | | | | 4.2 kb | | 4.2 kb | 4.2 kb | |
| UMC48-H | | | 3.5 kb | 3.5 kb | | | 3.5 kb | 3.5 kb | | | | 3.5 kb | | | | | | |
| UMC48-H | 2.2 kb | | | | | | | | | | | | | | | | | |
| UMC53-ERI | | 3.8 kb | 3.8 kb | 3.8 kb | | | 3.8 kb | 3.8 kb | 3.8 kb | | | 3.8 kb | | | | | | |
| UMC53-ERI | 3.0 kb | | | | | | | | | | | | | | | | | |
| npi268-B | 6.4 kb | 6.4 kb | 6.4 kb | 6.4 kb | | | 6.4 kb | 6.4 kb | 6.4 kb | 6.4 kb | 6.4 kb | 6.4 kb | | | | | | |
| UMC3-ERI | 2.5 kb | | 2.5 kb | 2.5 kb | | | 2.5 kb | 2.5 kb | | 2.5 kb | 2.5 kb | 2.5 kb | | | | | | |
| UMC3-ERI | 2.0 kb | 2.0 kb | | 2.0 kb | | | | | | | | | | | | | | |
| Chrom. 9 | | | | | | | | | | | | | | | | | | |
| phi10005-ERI | 15.0 kb | 15.0 kb | 15.0 kb | 15.0 kb | | | 15.0 kb | 15.0 kb | 15.0 kb | 15.0 kb | 15.0 kb | | | | | | | |
| phi10005-ERI | | | 1.6 kb | 1.6 kb | | | | | | | | | | | | | | |
| UMC113-ERI | 5.9 kb | | | | | | | | | | | | | | | | | |
| UMC113-ERI | | | 5.4 kb | 5.4 kb | | | | | | | | | | | | | | |
| UMC113-B | | | 12.8 kb | | | | | | | | | | | | | | | |
| UMC113-B | | 11.8 kb | | | | | | | | | | | | | | | | |
| UMC113-B | 10.5 kb | 10.5 kb | 10.5 kb | 10.5 kb | | | 10.5 kb | 10.5 kb | 10.5 kb | 10.5 kb | 10.5 kb | 10.5 kb | | | | | | |
| UMC192-H | 11.4 kb | 11.4 kb | 11.4 kb | 11.4 kb | | | 11.4 kb | 11.4 kb | 11.4 kb | 11.4 kb | 11.4 kb | 11.4 kb | | | | | | |
| UMC192-H | | 6.4 kb | | 6.4 kb | | | | | 6.4 kb | | | | | | | | | |
| UMC105-ERI | | | 3.9 kb | 3.9 kb | | | | | | | | | | | | | | |
| wx-H | 21.0 kb | | 21.0 kb | | | | | | | | | | | | | | | |
| CSU147-H | 5.9 kb | 5.9 kb | | 5.9 kb | | | | | | | | | | | | | | |
| BNL5.10-H | 6.1 kb | 6.1 kb | 6.1 kb | | | | | | | | 6.1 kb | | | | | | | |
| BNL5.10-H | 4.4 kb | | | 4.4 kb | | | | | | | | | | | | | | |
| UMC114-B | 15.0 kb | | 15.0 kb | 15.0 kb | | | | | | | | | | | | | | |
| UMC114-B | | | 12.6 kb | | | | | | | | 12.6 kb | | 12.6 kb | | | | | |
| UMC114-B | 11.5 kb | | | 11.5 kb | | | | | | | | | | | | | | |
| UMC114-B | | | 10.0 kb | 10.0 kb | | | 10.0 kb | 10.0 kb | | 10.0 kb | | 10.0 kb | | | | | | |
| UMC114-B | 8.8 kb | 8.8 kb | | 8.8 kb | | | | | 8.8 kb | | | | | | | | | |
| UMC114-B | | | 7.5 kb | 7.5 kb | | | | 7.5 kb | | 7.5 kb | | | | 7.5 kb | | 7.5 kb | | |
| UMC114-B | 6.5 kb | 6.5 kb | | 6.5 kb | | | | | | | | | | | | | | |
| UMC95-ERI | 13.3 kb | | 13.3 kb | | | 13.3 kb | | | | | | | | | | | | |
| UMC95-ERI | 5.6 kb | | 5.6 kb | | | | | | | | | | | | | | | |
| UMC95-H | | | 7.7 kb | | | | | | 7.7 kb | | | | | | | | | |
| UMC95-H | | | 7.3 kb | 7.3 kb | | | | | | | | | | 7.3 kb | | 7.3 kb | | |
| UMC95-H | 4.8 kb | 4.8 kb | 4.8 kb | | | | 4.8 kb | | 4.8 kb | | | | | | | | | |
| UMC95-H | | | 4.5 kb | 4.5 kb | | | | | | | | | | | | 4.5 kb | 4.5 kb | |
| UMC95-H | | | 4.1 kb | 4.1 kb | | | 4.1 kb | 4.1 kb | 4.1 kb | 4.1 kb | 4.1 kb | 4.1 kb | | | | | | |
| UMC95-H | | | 1.7 kb | 1.7 kb | | | | | | | | | | | | | | |
| UMC95-B | | | 15.0 kb | 15.0 kb | | | | | | | | | | | | | | |
| UMC95-B | | | 9.0 kb | 9.0 kb | | | | | | | | | | | | | | |
| asg44-ERI | | | 5.3 kb | 5.3 kb | | | | | | | | | | 5.3 kb | | 5.3 kb | | |
| CSU61-ERI | 8.1 kb | 8.1 kb | 8.1 kb | 8.1 kb | | | 8.1 kb | 8.1 kb | 8.1 kb | | 8.1 kb | 8.1 kb | | | | | | |
| CSU61-ERI | 4.8 kb | | 4.8 kb | | | | | | | 4.8 kb | | 4.8 kb | | | | | | |
| BNL7.57-ERI | 1.0 kb | | 1.0 kb | | | | | | | | | | | | | | | |
| BNL7.57-B | 11.6 kb | | | | | | | | | | | | | | | | | |
| BNL7.57-B | | 5.9 kb | | 5.9 kb | | | 5.9 kb | 5.9 kb | 5.9 kb | | | | | | | | | |
| CSU54-ERI | 14.7 kb | 14.7 kb | | 14.7 kb | | | | | 14.7 kb | | | | | | | | | |
| CSU54-ERI | | | 12.6 kb | | | | | 12.6 kb | | 12.6 kb | 12.6 kb | 12.6 kb | | | | | | |
| npi97-H | 3.9 kb | | 3.9 kb | | | | 3.9 kb | 3.9 kb | 3.9 kb | 3.9 kb | | | | | | | | |
| Chrom. 10 | | | | | | | | | | | | | | | | | | |
| phi20075-ERI | 7.1 kb | 7.1 kb | | 7.1 kb | | | | | 7.1 kb | | | | | | | | | |
| npi285-ERI | 15.3 kb | | 15.3 kb | | | | | | | | | | | | | | | |
| npi285-ERI | 12.4 kb | | 12.4 kb | | | | | | | 12.4 kb | 12.4 kb | | | | | | | |
| npi285-ERI | 9.4 kb | | | | | | | | | | | | | | | | | |
| npi285-ERI | | 6.0 kb | | 6.0 kb | | | | | 6.0 kb | | | | | | | | | |
| KSU5-ERI | 9.8 kb | 9.8 kb | | 9.8 kb | | | | | | | | | | | | | | |
| KSU5-ERI | | 7.6 kb | | | | | | | | | | | | | | | | |
| KSU5-ERI | | 6.1 kb | | | | | | | | | | | | | | | | |
| KSU5-ERI | | 3.8 kb | | | | | | | | | | | | | | | | |
| KSU5-ERI | 3.5 kb | | 3.5 kb | | | | | | | 3.5 kb | 3.5 kb | 3.5 kb | | | | | | |
| UMC130-ERI | | 13.5 kb | | 13.5 kb | | | | | | | | | | | | | | |
| UMC130-ERI | 7.0 kb | | 7.0 kb | 7.0 kb | | | 7.0 kb | 7.0 kb | 7.0 kb | 7.0 kb | 7.0 kb | 7.0 kb | | | | | | |
| UMC130-H | 4.8 kb | | 4.8 kb | 4.8 kb | | | 4.8 kb | 4.8 kb | 4.8 kb | 4.8 kb | 4.8 kb | 4.8 kb | | | | | | |
| UMC130-H | 3.2 kb | | 3.2 kb | 3.2 kb | | | | | 3.2 kb | | | | | | | | | |
| UMC130-B | 3.2 kb | | | | | | | | | | | | | | | | | |
| UMC64-H | 3.3 kb | | 3.3 kb | | | | | | | | | | | | | | | |
| UMC152-H | 12.4 kb | | | | | | | | | | | | | | | | | |
| UMC152-H | 7.1 kb | | 7.1 kb | | | | | | | 7.1 kb | 7.1 kb | | | | | | | |
| UMC152-H | | 5.6 kb | 5.6 kb | 5.6 kb | | | 5.6 kb | 5.6 kb | 5.6 kb | | | 5.6 kb | | | | | | |
| phi06005 | 12.8 kb | | 12.8 kb | 12.8 kb | | | 12.8 kb | 12.8 kb | | 12.8 kb | 12.8 kb | 12.8 kb | | | | | | |
| UMC163-H | | | 12.0 kb | 12.0 kb | | | | | | | | | | | | | | |
| UMC163-H | 7.0 kb | | | 7.0 kb | | | | | 7.0 kb | | | | | | | | | |
| UMC163-H | | 4.8 kb | 4.8 kb | 4.8 kb | | | 4.8 kb | 4.8 kb | 4.8 kb | 4.8 kb | 4.8 kb | | | | | | | |
| UMC163-H | | | | 3.0 kb | | | | | | | | | 3.0 kb | | | | | |
| UMC163-H | | | 2.3 kb | | | | | | | | | | | 2.3 kb | | | | |
| UMC44-H | | 9.8 kb | | | | | | | | | | | | | | | | |
| UMC44-H | | | 8.7 kb | 8.7 kb | | | | | | | 8.7 kb | 8.7 kb | | | | | | |
| UMC44-H | 7.2 kb | 7.2 kb | | | | | | | | | | | | | | | | |
| UMC44-H | | | 5.5 kb | 5.5 kb | | | 5.5 kb | 5.5 kb | | 5.5 kb | 5.5 kb | 5.5 kb | | 5.5 kb | | | | |
| UMC44-H | 4.0 kb | | | | | | | | | | | | | 4.0 kb | 4.0 kb | 4.0 kb | | 4.0 kb |
| BNL10.13-H | 10.8 kb | 10.8 kb | 10.8 kb | 10.8 kb | | | | | 10.8 kb | | | | | 10.8 kb | | | | |
| npi306-H | | | | 7.0 kb | | | | | | | | | | | | | | |
| Mitochondria | | | | | | | | | | | | | | | | | | |
| pmt1-H | | | | 2.3 kb | | | | | | | | | | | | | | |
| pmt2-H | 8.0 kb | 8.0 kb | 8.0 kb | 8.0 kb | | | | | | | | 8.0 kb | | | | | | |
| pmt2-H | | 4.2 kb | | | | | | | | | | | | | | | | |
| pmt2-H | 2.8 kb | 2.8 kb | 2.8 kb | 2.8 kb | | | | | 2.8 kb | 2.8 kb | 2.8 kb | | | | | | | |
| pmt2-H | 2.1 kb | | | | | | | | | | | | | | | | | |
| pmt5-H | 12.3 kb | 12.3 kb | 12.3 kb | 12.3 kb | | | | | | | | | | | | | | |
| pmt5-H | | 8.1 kb | | | | | | | | | | | | 8.1 kb | | | | |
| pmt5-H | | 3.2 kb | | 3.2 kb | | | | | | | | | | 3.2 kb | 3.2 kb | | | |
| pmt5-H | 2.5 kb | | 2.5 kb | 2.5 kb | | | | | | | | 2.5 kb | | | | | | |
| Unknown | | | | | | | | | | | | | | | | | | |
| tda16-H | | | | 4.3 kb | | | | | | | | | | | | | | |
| tda17-H | 7.0 kb | 7.0 kb | 7.0 kb | | | | 7.0 kb | 7.0 kb | 7.0 kb | 7.0 kb | 7.0 kb | 7.0 kb | | | | | | |
| tda48-H | | | 8.2 kb | 8.2 kb | | | | | | | | | | | | | | |
| tda53-H | 3.8 kb | | | | | | | | | | | | | | | | | |
| tda53-H | 2.2 kb | 2.2 kb | 2.2 kb | 2.2 kb | | | | 2.2 kb | 2.2 kb | 2.2 kb | 2.2 kb | 2.2 kb | | | | | | |
| tda168-ERI | 3.6 kb | | | 3.6 kb | | | | | | | | | | | | | | |
| tda250-B | | | 4.0 kb | | | | 4.0 kb | 4.0 kb | | | | 4.0 kb | | | | | | |

TABLE 3
Unique Tripsacum Alleles in Tripsacum-teosinte Hybrids and (Maize X Tripsacum-teosinte) Hybrids and Derivatives

| | Tripsacum-diploperennis Hybrids | | | | Maize X Tripsacum-diploperennis | | | | | | | | | | |
|---|---|---|---|---|---|---|---|---|---|---|---|---|---|---|---|
| Probe/ | Sun | | | Sun | | | | | | | Sun | | | | |
| Enzyme | Dance | 20A | Tripsacorn | Star | 2019 | 3024 | 3028 | 3125 | 4126 | TC64 | Devil | 7022 | 9094 | 97-5 | V70 |
| Chrom. 1 | | | | | | | | | | | | | | | |
| UMC107- | | 7.5 kb | | | | | | | | | | | | | |
| ERI | | | | | | | | | | | | | | | |
| Chrom. 2 | | | | | | | | | | | | | | | |
| UMC53- | 8.4 kb | | 8.4 kb | 8.4 kb | | | | 8.4 kb | 8.4 kb | | | | | | |
| ERV | | | | | | | | | | | | | | | |
| agrr167-B | | | 5.7 kb | 5.7 kb | | | | | | | | | | | |
| agrr167-B | | | | 4.5 kb | | | | | | | | | | | |
| agrr167-B | | | 4.0 kb | | | | | | | | | | | | |
| Chrom. 3 | | | | | | | | | | | | | | | |
| UMC50-B | 7.8 kb | 7.8 kb | | | | | | | | | | | | | |
| UMC50-B | 5.8 kb | | 5.8 kb | 5.8 kb | 5.8 kb | 5.8 kb | | 5.8 kb | 5.8 kb | 5.8 kb | | | | | |
| UMC42-H | 7.6 kb | | | 7.6 kb | | | | | | | | | | | |
| phi10080- | | | 9.7 kb | 9.7 kb | | | | | | | | | | | |
| B | | | | | | | | | | | | | | | |
| CSU25-H | | | 5.2 kb | 5.2 kb | | | | | | | | | 5.2 kb | 5.2 | |
| | | | | | | | | | | | | | | kb | |
| CSU25-H | 4.2 kb | 4.2 kb | | 4.2 kb | | | 4.2 kb | | | | | | | | |
| Chrom. 4 | | | | | | | | | | | | | | | |
| UMC31-B | 6.5 | | | | | | | | | | | | | | |
| phi20725- | 9.7 kb | | | | 9.7 kb | | 9.7 kb | | | | | | | | |
| ERI | | | | | | | | | | | | | | | |
| agrr89-H | | | 7.1 kb | 7.1 kb | | | | | | | | 7.1 kb | 7.1 kb | | 7.1 |
| | | | | | | | | | | | | | | | kb |
| npi386-H | | 12.6 kb | 12.6 kb | 12.6 kb | 12.6 kb | 12.6 kb | | | 12.6 kb | 12.6 kb | | | | | |
| UMC42-H | 7.6 kb | | | 7.6 kb | | | | | | | | | | | |
| tda62-B | 4.8 kb | 4.8 kb | | | | 4.8 kb | 4.8 kb | 4.8 kb | 4.8kb | | | | | | |
| Chrom. 5 | | | | | | | | | | | | | | | |
| asg73-ERI | | | 3.8 kb | 3.8 kb | | | | | | | | 3.8 kb | 3.8 kb | | 3.8 |
| | | | | | | | | | | | | | | | kb |
| UMC90-H | | 7.7 kb | 7.7 kb | 7.7 kb | 7.7 kb | 7.7 kb | 7.7 kb | 7.7 kb | | 7.7 kb | | | | | |
| UMC72 | | | 8.5 kb | 8.5 kb | | | | | | | | | | | |
| UMC27-H | 8.3 kb | | 8.3 kb | 8.3 kb | 8.3 kb | 8.3 kb | 8.3 kb | 8.3 kb | 8.3 kb | 8.3 kb | 8.3 | | | | |
| | | | | | | | | | | | kb | | | | |
| UMC43-B | 7.3 kb | 7.3 kb | 7.3 kb | 7.3 kb | 7.3 kb | 7.3 kb | | 7.3 kb | 7.3 kb | 7.3 kb | | | 7.3 kb | | |
| tda37-B | 9.0 kb | 9.0 kb | 9.0 kb | 9.0 kb | 9.0 kb | 9.0 kb | 9.0 kb | | | | | | | | |
| UMC40-B | | 4.7 kb | 4.7 kb | 4.7 kb | 4.7 kb | | | | | | | 4.7 kb | 4.7 kb | | 4.7 |
| | | | | | | | | | | | | | | | kb |
| Chrom. 6 | | | | | | | | | | | | | | | |
| NPI393- | 7.0 kb | 7.0 kb | | | | | | | | | | | | | |
| ERI | | | | | | | | | | | | | | | |
| UMC28-B | | | 9.9 kb | | | | | | 9.9 kb | | | | | | |
| Chrom. 7 | | | | | | | | | | | | | | | |
| asg8-H | | | | 8.7 kb | | | | | | | | | | | |
| phi20851- | | | 9.7 kb | 9.7 kb | | | | | | | | | | | |
| B | | | | | | | | | | | | | | | |
| UMC110- | 3.9kb | | | | | | 3.9 kb | 3.9 kb | 3.9 kb | | | | | | |
| B | | | | | | | | | | | | | | | |
| UMC80-H | 10.7 kb | 10.7 kb | 10.7 kb | 10.7 kb | 10.7 kb | 10.7 kb | 10.7 kb | 10.7 kb | 10.7 kb | 10.7 kb | | | | | |
| UMC80-H | 8.2 kb | 8.2 kb | 8.2 kb | 8.2 kb | | | | | 8.2 kb | | | | | | |
| BNL | 1.9 kb | | 1.9 kb | | | | | | | | | | | | |
| 16.06- | | | | | | | | | | | | | | | |
| ERI | | | | | | | | | | | | | | | |
| Chrom. 8 | | | | | | | | | | | | | | | |
| UMC48-H | | 6.2 kb | | 6.2 kb | | | 6.2 kb | | | | | | | | |
| UMC53- | 8.4 KB | | 8.4 KB | 8.4 KB | | | | 8.4 KB | 8.4 KB | | | | | | |
| ERV | | | | | | | | | | | | | | | |
| UMC7-B | 4.2 kb | | 4.2 kb | | | | | 4.2 kb | 4.2 kb | | | | | | |
| Chrom. 10 | | | | | | | | | | | | | | | |
| UMC163- | 2.6 kb | | | | | | 2.6 kb | | | | | | | | |
| H | | | | | | | | | | | | | | | |
| Mito- | | | | | | | | | | | | | | | |
| chondria | | | | | | | | | | | | | | | |
| pmt5-H | 3.6 kb | 3.6 kb | 3.6 kb | 3.6 kb | 3.6 kb | 3.6 kb | 3.6 kb | 3.6 kb | 3.6 kb | 3.6 kb | | | | | |
| Unknown | | | | | | | | | | | | | | | |
| tda168- | 3.6 kb | | | 3.6 kb | | | | | | | | | | | |
| ERI | | | | | | | | | | | | | | | |

TABLE 4
Restriction Fragment Sizes of Tripsacum and Teosinte Parent Plants

| | Parental RFLP Fragment Sizes | |
|---|---|---|
| Probe/Enzyme | Tripsacum | Z. diploperennis |
| Chromosome 1 | | |
| BNL5.62-ERI | Absent | 9.2 kb |
| npi97-H | 3.5 kb, 3.3 kb, 3.0 kb, 2.8 kb, 1.0 kb | 3.6 kb, 3.4 kb, 3.1 kb, 1.0 kb |
| UMC157-B | 10.2 kb, 3.8 kb | 6.7 kb, 5.0 kb, 3.8 kb |
| UMC11-B | Absent | 9.7 kb, 7.3 kb |
| asg45 | 3.2 kb | 1.7 kb |
| CSU3-B | Absent | 3.3 kb |
| UMC67-B | Absent | 9.8 kb |
| CSU92-B | Absent | Absent |
| asg62-B | Absent | 8.0 kb, 5.3 kb, 4.6 kb, 2.4 kb |
| UMC58-ERI | Absent | Absent |
| CSU164-ERI | 5.7 kb | 6.3 kb |
| UMC128-H | Absent | 10 kb |
| UMC107-ERI | 7.9 kb, 1.5 kb | 7.1 kb |
| UMC140-ERI | 10.9 kb, 7.5 kb | 2.6 kb |
| UMC161-B | 10.8 kb, 9.0 kb | 7.0 kb |
| BNL8.29-H | Absent | Absent |
| Chromosome 2 | | |
| UMC53-B | 8.7 kb | 3.8 kb |
| UMC53-ERV | 8.1 kb, 5.9 kb | 8.8 kb |
| UMC6-B | 11.5 kb, 3.3 kb | 11.5 kb, 8.2 kb, 3.3 kb |
| UMC61-H | 4.0 kb | 3.2 kb |
| agrr167-B | 4.0 kb | 4.4 kb |
| UMC34-ERI | Absent | 5.5 kb |
| UMC135-H | 12.5 kb, 11.4 kb | 12.5 kb, 11.4 kb, 9.0 kb |
| UMC131-ERI | 8.5 kb | 10.3 kb |
| UMC55-ERI | 4.3 kb, 3.7 kb | 3.7 kb, 1.8 kb |
| UMC5-ERI | 4.9 kb, 2.0 kb | 23.0 kb, 10.0 kb, 4.4 kb, 1.8 kb |
| tda66-ERI | 9.1 kb, 3.7 kb | 3.7 kb |
| UMC4-H | 4.5 kb | 8.0 kb, 5.4 kb |
| UMC49-B | 9.4 kb, 7.0 kb | 7.0 kb, 4.2 kb, 3.6 kb |
| UMC36-B | 10.5 kb, 9.4 kb, 8.2 kb, 7.8 kb, 6.6 kb | 3.9 kb |
| Chromosome 3 | | |
| UMC32-H | 4.2 kb | 9.8 kb |
| asg24-H | 4.4 kb | 6.8 kb |
| UMC121-ERI | Absent | 6.2 kb |
| BNL8.35-H | 3.1 kb | 12.2 kb, 10.0 kb |
| UMC50-B | 7.1 kb, 5.5 kb, 3.3 kb | 8.2 kb, 6.2 kb, 3.3 kb |
| UMC42-H | 9.2 kb, 7.6 kb, 3.3 kb, 2.7 kb | 3.4 kb |
| UMC10-H | 4.9 kb, 2.6 kb | 8.1 kb |
| UMC102-ERI | Absent | 8.3 kb, 6.6 kb, 2.4 kb, 1.4 kb |
| BNL6.06-ERI | Absent | 6.9 kb, 5.7 kb, 3.7 kb |
| BNL5.37-H | Absent | 9.7 kb, 5.7 kb |
| UMC3-ERI | 3.3 kb, 3.1 kb | 1.7 kb |
| UMC39-ERI | 5.7 kb, 2.6 kb, 1.7 kb, 1.6 kb | 11.5 kb, 7.0 kb, 5.7 kb, 2.5 kb |
| UMC15-B | 8.1 kb, 6.6 kb | 5.6 kb |
| UMC63-H | 8.7 kb, 7.4 kb, 7.0 kb, 5.9 kb | 14.1 kb,12.5 kb, 8.7 kb |
| UMC96-H | Absent | 8.7 kb, 4.3 kb, 3.1 kb, 2.9 kb |
| UMC2-ERI | 7.2 kb, 5.8 kb | 12.1 kb |
| CSU25-H | 9.9 kb, 5.2 kb, 4.0 kb, 3.0 kb, 2.0 kb | 5.9 kb, 4.6 kb |
| Chromosome 4 | | |
| phi20725-ERI | 9.8 kb, 5.9 kb | 5.9 kb |
| phi20725-H | 2.3 kb | 1.3 kb |
| UMC55-ERI | 4.3 kb, 3.7 kb | 3.7 kb, 1.8 kb |
| CSU235-H | 13.9 kb, 9.7 kb | 6.3 kb |
| CSU585-H | 9.5 kb, 7.1 kb, 5.7 kb, 3.7 kb | 7.6 kb, 5.4 kb, 4.1 kb, 3.7 kb, 3.0 kb |
| BNL5.46-H | 9.5 kb, 6.6 kb, 2.6 kb. 2.3 kb | 9.5 kb, 8.5 kb, 4.0 kb, 2.3 kb |
| npi386-H | 13.6 kb, 12.6 kb, 10.3 kb | 11.2 kb, 9.2 kb |
| UMC42-H | 9.2 kb, 7.4 kb, 3.3 kb, 2.7 kb | 3.5 kb |
| tda62-B | 4.8 kb, 3.7 kb, 1.8 kb, 1.4 kb | 9.5 kb, 6.7 kb, 5.1 kb, 4.7 kb, 2.5 kb, 1.4 kb |
| BNL5.71-ERV | 7.1 kb, 6.6 kb | 6.6 kb |
| UMC66-B | 7.0 kb, 3.7 kb | 10.5 kb, 3.7 kb |
| UMC19-B | 8.5 kb | 10.9 kb, 6.1 kb |
| UMC104-H | Absent | 7.1 kb, 6.7 kb |
| UMC133-H | Absent | 4.2 kb |
| UMC52-B | 11.8 kb, 5.7 kb | 13.9 kb, 4.1 kb, 3.6 kb |
| BNL15.07-H | Absent | 2.4 kb |
| Chromosome 5 | | |
| npi409-H | 13.0 kb, 8.4 kb, 3.0 kb | 13.0 kb, 4.6 kb |
| UMC147-H | Absent | 2.2 kb |
| asg73-ERI | 5.9 kb, 3.8 kb | 3.3 kb, 2.4 kb, 2.0 kb |
| UMC90-H | 8.4 kb, 7.7 kb, 5.0 kb | 2.4 kb, 2.2 kb |
| UMC107-ERI | 7.9 kb | 7.1 kb |
| UMC27-H | 11.8 kb, 8.0 kb | 5.0 kb |
| tda37-B | 9.0 kb | Absent |
| UMC43-B | Absent | 9.4 kb, 7.9 kb |
| UMC40-B | 6.1 kb, 5.2 kb, 2.7 kb | 4.2 kb, 3.2 kb |
| BNL7.71-H | 16.3 kb, 9.0 kb | 10.4 kb |
| UMC68-H | 13.3 kb, 5.8 kb, 5.1 kb, 4.3 kb | 5.8 kb, 5.1 kb |
| UMC104-B | Absent | 7.1 kb, 6.7 kb |
| Chromosome 6 | | |
| tda50-B | 10.8 kb, 6.8 kb, 6.6 kb | 8.0 kb |
| tda50-H | 1.7 kb | 1.4 kb |
| npi373-H | 9.0 kb | 9.0 kb, 6.2 kb |
| tda204-B | 14.4 kb, 10.5 kb, 9.9 kb | 7.3 kb, 0.9 kb |
| NPI393-ERI | 7.3 kb, 5.9 kb | 10.7 kb, 8.7 kb, 5.9 kb |
| UMC65-H | Absent | 3.0 kb |
| UMC21-ERI | Absent | 5.8 kb |
| UMC46-ERI | 13.6 kb, 11.9 kb, 11.1 kb, 8.4 kb | 5.9 kb, 5.1 kb |
| UMC132-H | 14.0 kb, 13.2 kb, 11.6 kb, 7.6 kb, 2.0 kb | 13.2 kb, 9.9 kb, 5.4 kb |
| asg7-H | Absent | 9.7 kb, 5.3 kb |
| UMC28-H | 10.0 kb | 5.8 kb, 2.0 kb |
| UMC28-B | 14.0 kb, 9.9 kb | 14.0 kb, 4.1 kb |
| UMC134-B | 9.9 kb, 2.9 kb, 2.8 kb, 2.7 kb | 4.2 kb, 3.6 kb |
| Chromosome 7 | | |
| asg8-H | 9.3 kb, 6.9 kb | 11.0 kb |
| BNL15.40-H | 6.8 kb, 3.9 kb, 3.2 kb | 10.4 kb, 5.1 kb |
| UMC116-ERI | Absent | Absent |
| UMC110-B | 7.3 kb, 6.6 kb, 3.9 kb | 7.3 kb |
| BNL8.32-H | 12.2 kb | 12.2 kb, 10.1 kb, 7.3 kb |
| BNL14.07-ERI | Absent | 6.7 kb, 5.7 kb |
| UMC80-H | 10.7 kb, 8.9 kb, 8.2 kb, 6.2 kb, 3.5 kb | 6.1 kb, 5.4 kb |
| BNL16.06-ERI | 8.6 kb, 7.2 kb, 3.1 kb, 2.0 kb | 8.6 kb, 6.7 kb, 3.7 kb, 1.8 kb |
| phi20020-H | 12.0 kb, 2.8 kb | 12.0 kb, 8.3 kb |
| Chromosome 8 | | |
| tda18-H | 7.7 kb, 7.0 kb, 2.9 kb | 6.1 kb |
| npi114-H | 5.4 kb, 3.9 kb | 3.9 kb, 1.3 kb |
| BNL9.11-H | 4.6 kb, 3.3 kb | 3.3 kb, 1.5 kb |
| UMC103-H | Absent | 11.5 kb |
| UMC124-B | 3.3 kb, 3.1 kb, 1.8 kb | 3.1 kb, 2.3 kb, 1.8 kb, 1.1 kb |
| UMC120-H | Absent | 2.1 kb, 1.5 kb |
| UMC89-B | Absent | 5.4 kb, 4.6 kb |
| BNL12.30-ERI | Absent | 8.9 kb |
| UMC48-H | 6.4 kb, 5.0 kb, 4.0 kb | 8.0 kb, 5.0 kb |
| UMC53-ERI | 8.7 kb, 8.2 kb | 3.8 kb |
| npi268-B | Absent | 6.8 kb, 6.2 kb |
| UMC7-B | 4.3 kb, 4.1 kb | 3.0 kb |
| UMC3-ERI | 3.3 kb, 3.2 kb, 3.1 kb | 1.7 kb |
| Chromosome 9 | | |
| phi10005-ERI | 6.1 kb | 10.2 kb |
| UMC113-ERI | 7.3 kb | Absent |
| UMC192-H | 10.7 kb, 9.9 kb, 9.2 kb, 1.7 kb | 8.3 kb, 7.3 kb, 2.1 kb |
| CSU147-H | 2.7 kb, 1.6 kb | 5.7 kb, 5.0 kb |
| BNL5.10-H | Absent | 2.5 kb |
| UMC114-B | Absent | 9.2 kb, 6.7 kb |
| UMC95-ERI | 4.1 kb | 4.8 kb, 4.1 kb |
| CSU61-ERI | 2.6 kb | 7.7 kb, 2.6 kb |
| BNL7.57-ERI | Absent | 5.0 kb, 4.4 kb |
| CSU54-ERI | 3.6 kb, 1.6 kb | Absent |
| Chromosome 10 | | |
| phi20075-ERI | 1.5 kb | 8.3 kb, 7.0 kb |
| npi285-ERI | 8.2 kb, 5.6 kb | 7.0 kb |
| KSU5-ERI | Absent | 3.5 kb, 2.2 kb |
| UMC130-ERI | Absent | Absent |
| UMC130-H | Absent | 8.8 kb, 4.3 kb |
| UMC152-H | Absent | 7.0 kb, 5.3 kb |
| phi06005 | 7.2 kb | 10.8 kb, 8.8 kb |
| UMC163-H | 6.6 kb, 6.4 kb, 5.7 kb, 2.8 kb | 12.1 kb, 4.6 kb, 4.2 kb |
| UMC44-H | 6.4 kb, 5.5 kb | 6.4 kb, 3.2 kb |
| BNL10.13-H | 12.6 kb, 9.1 kb, 6.7 kb, 6.0 kb | 3.9 kb |
| npi306-H | 2.3 kb, 2.0 kb | 11.3 kb, 9.0 kb |
| Mitochondria | | |
| pmt1-H | 7.5 kb, 6.5 kb | 8.4 kb, 2.8 kb, 2.7 kb |
| pmt2-H | 1.0 kb | 7.8 kb, 4.2 kb, 1.7 kb, 1.0 kb, 0.8 kb |
| pmt3-H | 2.9 kb, 2.3 kb | 5.1 kb, 2.1 kb |
| pmt4-H | 8.5 kb | 8.5 kb, 5.5 kb |
| pmt5-H | 7.4 kb, 3.8 kb, 2.7 kb | 9.1 kb, 5.9 kb, 4.4 kb, 3.6 kb |
| pmt6-H | 8.6 kb, 1.9 kb | 4.8 kb, 3.5 kb |
| Locus Unknown | | |
| tda16-H | 7.7 kb, 6.0 kb, 2.9 kb | 6.1 kb |
| tda17-H | 12.9 kb, 8.5 kb | Absent |
| tda48-H | 13.5 kb, 10.5 kb, 10.3 kb | 13.5 kb |
| tda53-H | 6.0 kb, 5.7 kb, 5.0 kb, 2.2 kb, 1.8 kb | 2.2 kb |
| tda168-ERI | 4.1 kb, 3.6 kb, 2.5 kb | 4.1 kb, 2.5 kb |
| tda250-B | 10.3 kb, 6.5 kb, 3.7 kb | 2.7 kb |

TABLE 5
RFLP and SSR Markers that Map to the Same Genetic Loci

| RFLP Marker | Corresponding SSR |
|---|---|
| Chromosome 1 | Chromosome 1 |
| BNL5.62 | bnlg1124 |
| npi97 | bnlg1112 |
| UMC157 | bnlg1953 |
| UMC76 | bnlg1484 |
| UMC11 | bnlg1083 |
| asg45 | bnlg1016 |
| CSU3 | bnlg2295 |
| UMC67 | bnlg1273 |
| asg62 | bnlg615 |
| UMC58 | bnlg1556 |
| UMC128 | bnlg1629 or bnlg2228 |
| UMC107 | bnlg1502 or bnlg1597 |
| adh1 | bnlg1268 |
| UMC161 | bnlg1671 |
| BNL8.29 | bnlg2331 |
| Chromosome 2 | Chromosome 2 |
| UMC53 | bnlg1338 or phi98 |
| UMC6 | bnlg125 or bnlg4696 |
| UMC61 | bnlg16216 |
| UMC34 | bnlg1064 |
| UMC135 | bnlg166 |
| UMC131 | bnlg1831 or bnlg1909 |
| UMC55 | bnlg1396 |
| UMC5 | bnlg1413 |
| UMC4 | bnlg1233 |
| UMC49 | bnlg1940 |
| Chromosome 3 | Chromosome 3 |
| asg24 | bnlg1523 |
| BNL8.35 | bnlg1047a or bnlg1798 |
| UMC10 | bnlg1452 |
| UMC102 | bnlg2047 |
| BNL5.37 | dupssr23 |
| UMC60 | bnlg2241 |
| UMC39 | bnlg1182 |
| UMC63 | bnlg1536 |
| UMC103 | bnlg1754 |
| UMC96 | bnlg1257 |
| UMC2 | bnlg1098 |
| Chromosome 4 | Chromosome 4 |
| agrr115 | bnlg372 or bnlg1370 |
| phi20725 | bnlg1241 |
| UMC87 | bnlg1126 |
| UMC31 | bnlg1162 |
| npi386 | bnlg1217 |
| UMC156 | bnlg1729 |
| UMC66 | bnlg2291 |
| UMC19 | dupssr34 |
| UMC15 | dupssr28 |
| UMC52 | bnlg1019b |
| BNL8.23 | bnlg1337 |
| BNL15.07 | bnlg589 |
| Chromosome 5 | Chromosome 5 |
| npi409 | bnlg1006 |
| UMC147 | bnlg1836 |
| UMC90 | bnlg143 or bnlg1382 |
| UMC72 | bnlg219 |
| UMC27 | bnlg1660 |
| BNL7.71 | bnlg1287 |
| BNL5/71 | bnlg2323 |
| UMC54 | bnlg609 or bnlg1246a |
| UMC108 | bnlg1306 |
| UMC68 | bnlg2305 |
| phi10017 | bnlg389 |
| Chromosome 6 | Chromosome 6 |
| UMC85 | bnlg426 |
| npi373 | bnlg1047b |
| UMC59 | bnlg2191 |
| NPI393 | bnlg2151 |
| UMC65 | phi124 |
| UMC21 | bnlg1922 or phi129 |
| UMC46 | bnlg1702 or bnlg2249 |
| UMC132 | bnlg1759a |
| asg7 | bnlg1521 |
| UMC28 | phi123 |
| Chromosome 7 | Chromosome 7 |
| asg8 | bnlg2132 |
| phi20581 | bnlg1292 |
| O2 | bnlg1200 |
| asg34 | bnlg1094 |
| BNL15.40 | bnlg1759b |
| UMC110 | bnlg572 |
| BNL8.32 | bnlg1805 |
| UMC80 | dupssr13 |
| BNL16.06 | bnlg23286 |
| phi20020 | phi69 |
| Chromosome 8 | Chromosome 8 |
| npi114 | bnlg2037 or bnlg1252 |
| BNL9.11 | bnlg1194 |
| UMC103 | bnlg2235 |
| UMC124 | bnlg1067 |
| UMC120 | bnlg669 |
| UMC89 | bnlg666 |
| Chromosome 9 | Chromosome 9 |
| phi10005 | bnlg2122 |
| UMC113 | phi122 |
| UMC105 | bnlg1082 |
| CSU147 | bnlg1626 |
| BNL5.10 | bnlg127 |
| UMC114 | bnlg469a |
| UMC95 | bnlg1714 |
| BNL5.09 | bnlg1588 |
| npi97 | bnlg1506 |
| Chromosome 10 | Chromosome 10 |
| UMC130 | bnlg1762 |
| UMC64 | bnlg2336 |
| phi06005 | bnlg1037 |
| tda205 | bnlg1074 |
| UMC163 | bnlg1185 |
| UMC44 | bnlg1250 |
| BNL10.13 | bnlg594 |
| npi306 | bnlg2190 |

Deposit of Seeds

A sample comprising at least 2500 seeds derived from crosses between Tripsacum dactyloides and Zea diploperennis as described herein were deposited with the American Type Culture Collection, 12301 Parklawn Drive, Rockville, Md. 20852 on Aug. 28, 1992. The accession number is ATCC75297.

The present invention is not limited in scope by the seeds deposited, since the deposited embodiments are intended as illustrations of the invention and any seeds, cell lines, plant parts, plants derived from tissue culture or seeds which are functionally equivalent are within the scope of this invention. An adequate supply of seed from other crosses, including crosses between Tripsacum laxum and Zea diploperennis, are available for deposit with the American Type Culture Patent Depository if necessary. While the invention has been described in detail and with reference to specific embodiments thereof, it will be apparent to one skilled in the art that changes and modifications can be made without departing from the spirit and scope of the invention in addition to those shown and described herein. Such modifications are intended to fall within the scope of the appended claims.

I claim:

1. A method of screening a plant to determine whether said plant is a cross between Tripsacum and teosinte, said method comprising the following steps:
(a) isolating the total genomic DNA from the plant in (c); then
(b) digesting said genomic DNA with one to five of the restriction enzymes selected from the group consisting of EcoRI, EcoRV, HindIII, BamHI and MspI; then
(c) probing said digested genomic DNA with one or more DNA markers selected from the group consisting of the maize nuclear DNA probes, maize mitochondrial DNA probes, and Tripsacum DNA probes recited below; and then
(d) determining the presence of one or more of the following restriction fragments of the following fragment sizes, wherein said restriction fragments are characterized by the following molecular marker-restriction enzyme associations and the associated fragment sizes selected from the group consisting of:
BNL5.62, EcoRI, 10.3 kb; npi97, HindIII, 3.9 kb; UMC157, EcoRI, 6.5 kb and 3.3 kb; UMC157, HindIII, 5.5 kb; UMC157, BamHI, 14.0 kb, 8.5 kb and 4.5 kb; UMC11, BamHI, 7.0 kb; CSU3, BamHI, 10.0 kb and 7.6 kb; UMC67, EcoRI, 19.2 kb; UMC67, BamHI 13.4 kb, 11.0 kb and 1.6 kb; CSU92, BamHI, 13.3 kb and 7.5 kb; asq62, BamHI, 12.7 kb, 9.7 kb and 6.6 kb; UMC58, HindIII, 3.3 kb; CSU164, EcoRI, 9.0 kb and 7.0 kb; UMC128, HindIII, 6.0 kb; UMC107, EcoRI, 7.5.0 kb, 6.3 kb and 6.1 kb; UMC140, EcoRI, 4.9 kb; UMC140, HindIII, 6.5 kb; adh1, HindIII, 9.4 kb; adh1, BamHI, 9.4 kb; UMC161, HindIII, 3.3 kb; BNL8.29, HindIII, 9.3 kb and 8.3 kb UMC53, EcoRI, 9.4 kb; UMC53, EcoRV, 8.4 kb, 3.8 kb and 3.0 kb; UMC6, EcoRI, 3.8 kb; UMC6, HindIII 9.4 kb; UMC6, BamHI, 13.2 kb, 12.7 kb, and 7.0 kb; UMC61, HindIII, 3.4 and 2.8 kb agrr167, BamHI, 5.7 kb, 4.5 kb and 4.0 kb; UMC34, EcoRI, 7.5 kb and 5.4 kb; UMC34, HindIII, 8.8 kb, 6.5 kb and 5.8 kb; UMC34, BamHI, 9.4 kb; UMC135, HindIII, 11.6 kb and 10.8 kb; UMC131, EcoRI, 10.6 kb, 5.8 kb and 4.3 kb; UMC55, EcoRI, 3.9 kb; UMC55, HindIII, 4.3 kb; UMC5, EcoRI, 5.4 kb; UMC5, HindIII, 6.5 kb; UMC49, BamHI, 8.2 kb; UMC36, BamHI, 4.2 kb; UMC32, EcoRI, 5.3 kb; UMC32, HindIII 6.7 kb, 6.0 kb, and 2.8 kb; asq24, HindIII, 7.2 kb and 6.4 kb; UMC121, EcoRI, 3.7 kb and 3.2 kb; BNL8.35, HindIII, 9.9 kb and 8.7 kb; UMC50, BamHI, 7.8 kb, 6.8 kb, 5.8 kb and 3.8 kb; UMC42, HindIII, 10.4 kb, 9.2 kb, 8.9 kb, 7.9 kb, 7.6 kb, and 3.7 kb; npi247, EcoRI, 8.0 kb; npi247, HindIII 3.0 kb; UMC10, HindIII, 3.0 kb; UMC10, EcoRI, 6.5 kb and 5.5 kb; UMC102, EcoRI, 2.7 kb; BNL6.06, EcoRI, 6.8 kb; CSU240, EcoRI, 10.6 kb, 4.5 kb and 3.3 kb; BNL5.37, HindIII, 10.3 kb, 5.8 kb and 3.5 kb; npi296, EcoRI, 7.9 kb; UMC3, EcoRI 2.5 kb and 2.0 kb; npi212, HindIII, 4.3 kb; npi212, BamHI, 5.4 kb; UMC39, EcoRI, 12.2 kb, 9.2 kb, 7.8 kb and 7.1 kb; phi10080, BamHI, 9.7 kb; UMC63, HindIII, 9.5 kb and 4.3 kb; CSU303, EcoRI, 10.0 kb; UMC96, HindIII, 11.8 kb, 6.4 kb and 5.5 kb; UMC96, BamHI, 7.5 kb; UMC2, EcoRI, 11.8 kb, 10.4 kb, 8.0 kb and 3.9 kb; CSU25, HindIII, 5.2 kb, 4.5 and 4.2 kb; agrr115, EcoRI. 8.0 kb and 5.4 kb; agrr115, BamHI, 5.4 kb and 3.5 kb; phi20725, EcoRI, 10.3 kb, 9.7 kb and 7.2 kb; phi20725, HindIII, 1.5 kb; UMC31, EcoRI, 5.8 kb and 2.0 kb; UMC31, BamHI 6.5 kb; UMC55, EcoRI, 3.9 kb; UMC55, HindIII, 4.3 kb; CSU235, HindIII, 6.8 kb and 3.0 kb; CSU585, HindIII, 8.3 kb and 6.1 kb; BNL5.46, HindIII, 13.7 kb, 10.5 kb, 9.7 kb and 5.1 kb; aarr321, BamHI, 5.5 kb; agrr89, HindIII, 7.1 kb; npi386, HindIII, 12.6 kb, 9.3 kb and 8.2 kb; UMC42, HindIII, 19.2 kb, 10.3 kb 8.9 kb, 7.6 kb, 3.7 kb and 3.0 kb; tda62, BamHI, 5.5 kb, 5.2 kb, 4.8 kb and 4.2 kb; BNL5.71, EcoRV, 11.3 kb, 6.8 kb, and 5.7 kb; UMC156, HindIII, 3.0 kb; UMC66, EcoRI, 10.5 kb; UMC66, BamHI, 3.7 kb and 2.4 kb; UMC19, BamHI, 12.3 kb; UMC104, HindIII, 12.4 kb, 11.6 kb and 7.5 kb; UMC104, BamHI, 9.4 kb; UMC133, HindIII, 10.6 kb, 9.9 kb, 9.2 kb and 7.7 kb; UMC52, BamHI, 8.7 kb, 6.9 kb, 3.8 kb, 3.0 kb and 2.0 kb; BNL15.07, HindIII, 2.9 kb and 2.7 kb; npi409, EcoRI, 9.4 kb; npi409, HindIII, 10.4 kb, 9.0 kb and 3.9 kb; UMC147, HindIII, 16.3 kb, 3.8 kb and 2.4 kb; asg73, EcoRI, 3.8 kb; UMC90, HindIII, 7.7 kb, 6.5 kb, 2.8 kb and 1.6 kb; UMC90, BamHI, 9.0 kb; UMC72, 8.5 kb; UMC27, HindIII, 8.3 kb and 4.5 kb; UMC27, BamHI, 6.5 kb; UMC43, BamHI, 9.7 kb, 7.3 kb and 5.7 kb; tda37, BamHI, 9.0 kb, 8.0 kb and 6.4 kb; UMC43, BamHI, 9.7 kb, 7.3 kb and 5.7 kb; UMC40, BamHI, 7.2 kb, 4.7 kb and 4.3 kb; BNL7.71, HindIII, 10.6 kb; BNL5.71, BamHI, 11.3 kb, 6.8 kb and 5.7 kb; tda62, BamHI, 6.5 kb and 5.5 kb; UMC68, HindIII, 6.0 kb; UMC104, HindIII, 12.4 kb, 11.6 kb and 7.5 kb; UMC104, BamHI, 9.4 kb; phi10017, BamHI, 15.1 kb and 9.5 kb; tda50, BamHI, 8.5 kb; npi373, HindIII, 6.5 kb, 5.6 kb, 5.1 kb and 3.0 kb; tda204, BamHI, 4.0 kb; npi393, EcoRI, 12.1 kb, 8.5 kb, 7.0 kb and 5.6 kb; UMC65, HindIII, 2.9 kb; UMC46, EcoRI, 6.5 kb and 5.6 kb; asg7, HindIII, 6.3 kb; UMC28, HindIII, 15.8 kb and 11.9 kb; UMC28, BamHI, 9.9 kb, 7.6 kb and 6.6 kb; UMC134, HindIII, 7.5 kb and 4.7 kb; asg8, HindIII, 10.8 kb, 8.7 kb and 8.4 kb; phi20581, HindIII, 4.2 kb; O2, EcoRI, 9.4 kb; asg34, HindIII, 4.5 kb; BNL15.40, HindIII, 5.8 kb; UMC116, EcoRI, 9.5 kb; UMC110, BamHI, 10.6 kb, 4.9 kb and 3.9 kb; BNL8.32, HindIII, 8.9 kb, 7.4 kb and 7.1 kb; BNL14.07, EcoRI, 6.4 kb; UMC80, HindIII, 10.7 kb, 8.2 kb and 2.4 kb; BNL16.06, EcoRI, 6.8 kb and 1.9 kb; BNL16.06, HindIII, 5.7 kb, 3.0 kb and 1.6 kb; phi20020, HindIII, 7.8 kb, 6.6 kb and 5.1 kb; npi114, HindIII, 10.0 kb, 8.8 kb and 6.3 kb; BNL9.11, HindIII, 3.4 kb; UMC103, HindIII, 6.9 kb; UMC124, HindIII, 8.0 and 7.0; UMC124, BamHI, 6.6 kb, 2.6 kb and 1.6 kb; UMC120, HindIII, 3.2 kb, 2.3 kb and 1.4 kb; UMC89, EcoRI, 7.3 kb; UMC89, HindIII, 7.3 kb; UMC89, BamHI, 9.5 kb, 6.0 kb, 5.2 kb and 4.5 kb; UMC89, MspI, 6.7 kb and 5.8 kb; BNL12.30, EcoRI, 3.5 kb; UMC48, HindIII, 6.2 kb, 5.3 kb, 4.7 kb, 4.2 kb and 3.5 kb; UMC53, EcoRI, 3.8 kb and 3.0 kb; UMC53, EcoRV, 8.4 kb; npi268, BamHI, 6.4 kb; UMC7, BamHI, 4.2 kb; UMC3, EcoRI, 3.5 kb and 2.0 kb; phi10005, EcoRI, 15.0 kb and 1.6 kb; UMC113, EcoRI, 5.9 kb and 5.4 kb; UMC113, BamHI, 12.8 kb, 11.8 kb and 10.5 kb; UMC192, HindIII, 11.4 kb and 6.4 kb; wx (waxy), HindIII, 21.0 kb; UMC105, EcoRI, 3.9 kb; CSU147, HindIII 5.9 kb; BNL5.10, HindIII, 6.1 kb and 4.4 kb; UMC114, BamHI, 12.6 kb, 11.5 kb, 10.0 kb, 8.8 kb, 7.5 kb and 6.5 kb; UMC95, EcoRI, 5.6 kb; UMC95, HindIII, 7.7 kb, 7.3 kb, 4.8 kb, 4.5 kb 4.1 kb and 1.7 kb; UMC95, BamHI, 15.0 kb and 9.0 kb; asa44, EcoRI, 5.3 kb; CSU61, EcoRI, 8.1 kb and 4.8 kb; BNL7.57, BamHI, 11.6 kb and 5.9 kb; CSU54, EcoRI, 14.7 kb and 12.6 kb; phi20075, EcoRI, 7.1 kb; npi285, EcoRI, 12.4 kb, 9.4 kb and 6.0 kb; KSU5, EcoRI, 9.8 kb, 7.6 kb, 6.1 kb, 3.8 kb and 3.5 kb; UMC130, EcoRI, 13.5 kb and 7.0 kb; UMC130, HindIII, 4.8 kb and 3.2 kb; UMC130, BamHI, 3.2 kb; UMC64, HindIII, 3.3 kb; UMC152, HindIII, 12.4 kb, 7.1 kb and 5.6 kb; phi06005, EcoRI, 12.8 kb; UMC163, HindIII, 7.0 kb, 4.8 kb; 3.0 kb; 2.6 kb and 2.3 kb; UMC44, HindIII, 9.8 kb, 8.7 kb, 7.2 kb, 5.5 kb and 4.0 kb; BNL10.13, HindIII, 10.8 kb; npi306, HindIII, 7.0 kb; pmt1, HindIII, 2.3 kb; pmt2, HindIII, 2.8 kb and 2.1 kb; pmt5, HindIII, 12.3 kb, 8.1 kb, 3.6 kb, 3.2 kb and 2.5 kb; tda48, HindIII, 8.2 kb; tda53, HindIII, 3.8 kb and 2.2 kb; tda168, EcoRI, 3.6 kb; tda16, HindIII, 4.3 kb; and tda17, HindIII, 7.0 kb; tda250, BamHI, 4.0 kb.

2. A plant containing one or more novel restriction fragments identified by one or more molecular marker-enzyme combinations in claim 1 thereof, produced from a procedure comprising the steps of:
(a) crossing a Tripsacum female parent with a teosinte male parent to produce (Tripsacum X teosinte) hybrid seed or a teosinte female parent with a Tripsacum pollen donor to produce (teosinte X Tripsacum) hybrid seed; then
(b) growing a (Tripsacum X teosinte) or (teosinte X Tripsacum) hybrid plant from said seed to maturity; then
(c) harvesting the seed produced in (c).

3. Seed from a plant in claim 2 that contains one or more restriction fragments produced in accordance with the method described in claim 1.

4. All hybrid plants, derivatives, variants, mutants, modifications, and cellular and molecular components that contain one or more restriction fragments set forth in claim 1 thereof, obtained from a plant as set forth in claim 2 or grown from seed according to claim 3.

5. Pollen produced by a plant according to claims 2 or 4 that contains one or more restriction fragments described in claim 1.

6. A tissue culture, all derivatives, variants, mutants, modifications, and cellular and molecular components from a plant according to claim 4 that contain one or more restriction fragments described in claim 1.

7. A method of screening a plant in accordance with claim 1 wherein said plant is a maize plant that contains one or more restriction fragments described in claim 1 thereof.

8. A plant wherein said plant is a maize plant that contains one or more restriction fragments described in claim 1 thereof, and is produced from a procedure comprising the steps of:
(a) crossing a Tripsacum female parent with a teosinte male parent to produce (Tripsacum X teosinte) hybrid seed or a teosintz female parent with a Tripsacum pollen donor to produce (teosinte X Tripsacum) hybrid seed; then
(b) growing a (Tripsacum X teosinte) or (teosinte X Tripsacum) hybrid plant from said seed to maturity; then
(c) crossing said seed from (Tripsacum X teosinte) or (teosinte X Tripsacum) hybrid plant with maize to produce seed;
(d) harvesting the seed produced in (c).

9. Maize seed that contains one or more restriction fragments described in claim 1 thereof, produced from a plant in claim 8.

10. Maize plants, all derivatives, subsequent generations, variants, mutants, modifications, and cellular and molecular components that contain one or more restriction fragments described in claim 1 thereof, grown from said seed according to claim 9.

11. Pollen that contains one or more restriction fragments described in claim 1 thereof, produced by a plant according to claim 8 or claim 10.

12. Tissue cultures, all derivatives, variants, mutants, modifications, and cellular and molecular components that contain one or more restriction fragments described in claim 1 thereof, derived from said hybrid maize plants according to claim 8 or claim 10.
